SEQUENCE LISTING (6 sequences)

SEQ ID NO: 1 - DNA, 1176 bp, Artificial Sequence
  GITR Chimeric Construct
atggcacagcacggggcgatgggcgcgtttcgggccctgtgcggcctggcgctgctgtgcgcgctcagcctgggtcagcgccccaccgggggtcccgggtgcggccctgggcgcctcctgcttgggacgggaacggacgcgcgctgctgccgggttcacacgacgcgctgctgccgcgattacccgggcgaggagtgctgttccgagtgggactgcatgtgtgtccagcctgaattccactgcggagacccttgctgcacgacctgccggcaccacccttgtcccccaggccagggggtacagtcccaggggaaattcagttttggcttccagtgtatcgactgtgcctcggggaccttctccgggggccacgaaggccactgcaaaccttggacagactgcacccagttcgggtttctcactgtgttccctgggaacaagacccacaacgctgtgtgcgtcccagggtccccgccggcagaggcggccgcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 391 aa, Artificial Sequence
  GITR Chimeric Construct
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEAAADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 1116 bp, Artificial Sequence
  GITR Chimeric Construct
atggactttgggctcagcttcattttccttgcccttattttaaaaggtgtccagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtgccactgctaaggagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctag

SEQ ID NO: 4 - protein, 371 aa, Artificial Sequence
  GITR Chimeric Construct
MDFGLSFIFLALILKGVQCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 5 - DNA, 1134 bp, Artificial Sequence
  GITR Chimeric Construct
atggactttgggctcagcttcattttccttgcccttattttaaaaggtgtccagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtgcctcactcaagccaactgccatcgagtcctgcatggttaagtttgaactatcatcctcaaaatggcacatgacatctcccaaacctcactgtgtgaatacgacatctgatgggaagctgaagatactgcagagtggcacatatttaatctacggccaagtgattcctgtggataagaaatacataaaagacaatgcccccttcgtagtacagatatataaaaagaatgatgtcctacaaactctaatgaatgattttcaaatcttgcctataggaggggtttatgaactgcatgctggagataacatatatctgaagttcaactctaaagaccatattcagaaaactaacacatactgggggatcatcttaatgcctgatctaccattcatctcttag

SEQ ID NO: 6 - protein, 377 aa, Artificial Sequence
  GITR Chimeric Construct
MDFGLSFIFLALILKGVQCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGASLKPTAIESCMVKFELSSSKWHMTSPKPHCVNTTSDGKLKILQSGTYLIYGQVIPVDKKYIKDNAPFVVQIYKKNDVLQTLMNDFQILPIGGVYELHAGDNIYLKFNSKDHIQKTNTYWGIILMPDLPFIS

(19) United States
(12) Patent Application Publication
(10) Pub. No.: US 2019/0300619 A1
(43) Pub. Date: Oct. 3, 2019

(54) GITR AGONISTS, AND METHODS OF USE THEREOF

(71) Applicant: University of Southern California, Los Angeles, CA (US)

(72) Inventors: Omid Akbari, Los Angeles, CA (US); Peisheng Hu, Los Angeles, CA (US); Alan Epstein, Los Angeles, CA (US)

(21) Appl. No.: 16/358,522

(22) Filed: Mar. 19, 2019

Related U.S. Application Data
(60) Provisional application No. 62/644,870, filed on Mar. 19, 2018.

(51) Int. Cl. C07K 16/28; A61K 38/17; A61P 3/10

(52) U.S. Cl. CPC C07K 16/2878; A61P 3/10; A61K 38/177

(57) ABSTRACT

Presented herein, in certain embodiments, are compositions comprising a GITR agonist and uses thereof for the treatment of diabetes or related disorders.

### RELATED PATENT APPLICATIONS

This patent application claims the benefit of U.S. Provisional Patent Application No. 62/644,870 filed on Mar. 19, 2018, entitled GITR AGONISTS, AND METHODS OF USE THEREOF, naming Omid Akbari, Peisheng Hu and Alan Epstein as inventors, and designated by Attorney Docket No. 043871-0458524. The entire content of the foregoing application is incorporated herein by reference, including all text, tables and drawings.

### FIELD OF THE INVENTION

Embodiments of the invention relate to agonist of the Glucocorticoid-induced Tumor Necrosis Factor Receptor (GITR), compositions thereof and uses thereof for the prevention and/or treatment of diabetes or a related disorder.

### INTRODUCTION

Chronic obesity is associated with the development of low-grade systemic inflammation and recruitment of inflammatory immune cells to metabolic active tissues, such as the adipose tissue, liver, and muscle. This pro-inflammatory environment promotes insulin resistance and elevation of blood glucose levels, predisposing patients to development of Type-2 Diabetes. The metabolic disturbances associated with obesity-induced inflammation in the adipose tissue are thought to involve the expansion and a remodeling of immune cells, particularly in visceral adipose tissue (VAT). In this regard, type 2 innate lymphoid cells (ILC2s) are among the various immune cells that have recently been identified to be present in the VAT.

Presented herein are compositions and methods that can be used to prevent and/or treat diabetes, obesity, insulin resistance and related disorders by, in certain embodiments, modulating amount and activity of type 2 innate lymphoid cells.

### SUMMARY

In some aspects, presented herein is a method of preventing or treating diabetes (e.g., Type-2 Diabetes Mellitus), or a related disorder in a subject comprising: providing a subject having, suspected of having, or at risk of having diabetes, or a related disorder, and administering a therapeutically effective amount of a Glucocorticoid-Induced Tumor Necrosis Factor Receptor (GITR) agonist. In some aspects, presented herein is a method of regulating blood glucose levels in a subject comprising administering a therapeutically effective amount of a GITR agonist to the subject. In some aspects, presented herein is a method of treating or preventing insulin resistance in a subject comprising: providing a subject having, suspected of having, or at risk of having insulin resistance and administering a therapeutically effective amount of a GITR agonist to the subject.

In some aspects, presented herein is a method of improving glucose tolerance and/or insulin sensitivity in a subject comprising administering a therapeutically effective amount of a GITR agonist to the subject.

In some aspects, presented herein is a method of preventing, or preventing the onset of, or treating diabetes (e.g., Type-2 Diabetes Mellitus), or related disorder in a subject comprising contacting a type-2 innate lymphoid cell with a GITR agonist. In some aspects, presented herein is a method of regulating blood glucose levels in a subject comprising contacting a type-2 innate lymphoid cell with a GITR agonist. In some embodiments, the regulating of blood glucose levels in the subject comprises maintaining blood glucose levels within a normal healthy range, lowering blood glucose levels to a normal healthy range or preventing elevation of blood glucose levels to an abnormal, unhealthy range. In some aspects, presented herein is a method of treating or preventing insulin resistance in a subject comprising contacting a type-2 innate lymphoid cell with a GITR agonist. In some aspects, presented herein is a method of improving glucose tolerance and/or insulin sensitivity in a subject comprising contacting a type-2 innate lymphoid cell with a GITR agonist. In some aspects, presented herein is a method of inducing production of, and/or secretion of, one or more Th2-cytokines from a type-2 innate lymphoid cell comprising contacting the type-2 innate lymphoid cell with a GITR agonist. In some aspects, presented herein is a method of inducing production of, and/or secretion of, IL-5, IL-13, GM-CSF, IL-6 or IL-9 in or from a type-2 innate lymphoid cell comprising contacting the type-2 innate lymphoid cell with a GITR agonist. In some aspects, presented herein is a method of activating a type-2 innate lymphoid cell comprising contacting the type-2 innate lymphoid cell with a GITR agonist. In some embodiments, the activating comprises increasing production and/or secretion of TH2 cytokines. In some aspects, presented herein is a method of inducing NF-kB pathway signaling in a type-2 innate lymphoid cell comprising contacting the type-2 innate lymphoid cell with a GITR agonist. In some embodiments, NF-kB signaling is anti-inflammatory. In some aspects, presented herein is a method of increasing the amount or cell numbers of a type-2 innate lymphoid cell in adipose tissue comprising contacting the a type-2 innate lymphoid cell with a GITR agonist. In some embodiments, the adipose tissue is visceral adipose tissue (VAT). In some aspects, presented herein is a method of protecting a subject against obesity-induced metabolic disturbances comprising contacting a type-2 innate lymphoid cell with a GITR agonist. In some aspects, presented herein is a method of modulating macrophage polarization comprising contacting a type-2 innate lymphoid cell with a GITR agonist. In some aspects, presented herein is a method of ameliorating established diabetes (e.g., Type-2 Diabetes), or a related disorder, in a subject comprising contacting a type-2 innate lymphoid cell with a GITR agonist.

In some embodiments, a GITR agonist specifically binds to a GITR. In some embodiments, a GITR agonist activates cell signaling through the GITR and/or activates NF-kB. In some embodiments, a GITR is expressed on, or located on, the surface of the type-2 innate lymphoid cell. In some embodiments, a GITR agonist is an antibody, antigen binding fragment thereof, or antibody-like agent that specifically binds to GITR. In some embodiments, the antibody is a monoclonal antibody. In some embodiments, the GITR agonist comprises DTA-1, or a humanized version of DTA-1. In some embodiments, a GITR agonist comprises a GITR ligand (GITRL), or a variant or derivative thereof.

In some aspects the type-2 innate lymphoid cell is located in a subject. In some embodiments, a subject is a mammal. In some embodiments, the mammal is a human or a mouse. In some embodiments, the subject has, is suspected of having, or is at risk of having diabetes (e.g., Type-2 Diabetes Mellitus), insulin resistance, elevated blood glucose levels, obesity, or a combination thereof. In some embodiments, the method comprises administering a therapeutically effective amount of the GITR agonist to the subject.

In some embodiments, a type-2 innate lymphoid cell is obtained from, or derived from a subject.

In some embodiments, a GITR agonist can be administered in combination with a therapeutically effective amount of a second active agent (e.g., an API) for the treatment of diabetes or a related disorder. A GITR agonist and a second active agent can be administered at the same time or at different times. In some embodiments, a second active agent comprises a pharmaceutical drug known to be effective for the treatment or prevention of diabetes or a related disorder. In some aspects the methods disclosed herein comprise contacting a type-2 innate lymphoid cell with a GITR agonist and IL-33 or IL-25. In some aspects the methods disclosed herein comprise contacting a type-2 innate lymphoid cell with a GITR agonist and IL-2 or IL-7. Accordingly, in some aspects a subject having, suspected of having or at risk of having diabetes, or a related disorder is administered a GITR agonist and one or more additional therapeutic agents which are, in certain embodiments, selected from IL-33, IL-25, IL-2, and IL-7.

Certain aspects of the technology are described further in the following description, examples, claims and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1—GITR is expressed on ILC2s and its engagement induces Th2-cytokine secretion in activated ILC2s. FIG. 1A shows a gating strategy of Lin⁻CD45⁺IL-7R⁺ST2⁺ILC2 cells. FIG. 1B shows expression of GITR in naïve (gray) and activated (red) murine white adipose tissue-derived ILC2s compared to the isotype control (white). Corresponding quantitation of GITR expression is shown as MFI+/−SEM. FIG. 1C shows adipose tissue (AT) resident ILC2s isolated from a cohort of C57BL/6 mice and stimulated with recombinant mouse (rm) IL-2 and rmIL-7 with or without DTA-1 (5 μg/mL) for 48 h. The levels of IL-5, IL-13, GM-CSF, IL-6 and IL-9 were measured by Luminex on the culture supernatants. FIG. 1D shows a cohort of C57BL/6 mice intranasally challenged with rmIL-33 on days 1-3. On day 4 ILC2s from AT were isolated and re-stimulated with rmIL-2 and rmIL-7 with or without DTA-1 (5 μg/mL) for 48 h. The levels of IL-5, IL-13, GM-CSF, IL-6 and IL-9 were measured by Luminex on the culture supernatants.

FIG. 2—Engagement of GITR protects from the onset of Type-2 Diabetes. FIG. 2A shows a cohort of Ob/Ob mice treated with PBS, DTA-1 (1 mg/mice) or rmL-33 (0.5 μg/mice) by intraperitoneal injections every four days according to the scheme. FIG. 2B and FIG. 2C shows total weight and fasting blood glucose levels, respectively, measured every two weeks for 14 weeks. FIG. 2D and FIG. 2E shows a glucose tolerance test and insulin tolerance test, respectively, performed in a cohort of Ob/Ob mice after 14 weeks of treatment. The area under the curve was calculated for each group. Mice were euthanized on 0, 3, 6 and 10 weeks and AT (adipose tissue) resident ILC2s were quantified at the indicated times (FIG. 2F). Representative FACS plots of AT Lin⁻CD45⁺IL-7R⁺ST2⁺ILC2s on week 10 are shown in FIG. 2G.

FIG. 3—Preventive GITR engagement protects from Type-2 Diabetes in Rag2^{-/-} mice.

FIG. 3A shows a cohort of Rag2^{-/-} mice fed on High Fat Diet (HFD) and either treated with PBS or DTA-1 (1 mg/mice) by intraperitoneal injection every four days according to the scheme. FIG. 3B and FIG. 3C shows total weight and blood glucose levels, respectively, measured every two weeks for 14 weeks. FIG. 3D shows plasma insulin concentration measured by ELISA. FIG. 3E and FIG. 3F show a glucose tolerance test and insulin tolerance test, respectively, performed in a cohort of mice after 14 weeks of treatment. The area under the curve was calculated for each group. FIG. 3G shows a hematoxylin and eosin-stained epididymal adipose tissue sections (×400). Red arrows point to recruited inflammatory cells around adipocytes, scale bars, 100 μm. FIG. 3H shows a corresponding quantitation presented as the mean adipocyte area. FIGS. 3I, 3J, and 3K) show a CLAMS analysis performed using individually housed groups of PBS, DTA-1 and IL-33 treated C57BL/6 mice maintained on a HFD. Frequency of body fat (FIG. 3I), variations in oxygen consumption (FIG. 3J) and energy expenditure (FIG. 3K) were measured. Data were pooled among animals in each group.

FIG. 4—GITR engagement drives ILC2-derived Th2 cytokine secretion and M2 macrophage polarization. A cohort of Rag2^{-/-} mice were fed on HFD and either treated with PBS, DTA-1 (1 mg/mice) or rmIL-33 (0.5 μg/mice) by intraperitoneal injection every four days.

FIG. 4A shows representative flow cytometry plots of AT Lin⁻CD45⁺IL-7R⁺ST2⁺ILC2s in each group after 14 weeks of treatment, and corresponding quantitation presented as the number of ILC2s per gram of VAT. FIG. 4B shows representative flow cytometry plots of intracellular IL-5 (top panel) and IL-13 (bottom panel) in AT ILC2s and corresponding quantitation after 14 weeks of treatment, presented as frequency of positive cells relative to PBS-treated mice. FIG. 4C shows representative flow cytometry plots of CD45⁺CD11b^{hi}F4/80^{hi}CD206⁺CD11c⁺ M2 macrophages in AT and corresponding quantitation after 14 weeks of treatment.

FIG. 5—Therapeutic GITR treatment ameliorates established Type-2 Diabetes in Rag2^{-/-} mice. FIG. 5A shows a cohort of Rag2^{-/-} mice fed on HFD for 14 weeks. After 8 weeks, mice were either treated with PBS, DTA-1 (1 mg/mice) or recombinant mouse IL-33 (0.5 μg/mice) by intraperitoneal injection every four days according to the scheme. FIGS. 5B and C shows total weight (FIG. 5B) and fasting blood glucose levels (FIG. 5C) measured every two weeks for 14 weeks. FIG. 5D shows plasma insulin concentrations measured by ELISA. FIGS. 5E and 5F show the results of a glucose tolerance test (FIG. 5E) and insulin tolerance test (FIG. 5F) performed in a cohort of mice after 8 weeks of treatment. The area under the curve was calculated for each group. FIG. 5G shows hematoxylin and eosin-stained epididymal adipose tissue sections (×400). Red arrows point to recruited inflammatory cells around adipocytes, scale bars, 100 μm. FIG. 5H shows corresponding quantitation presented as the mean adipocyte area.

FIG. 6—GITR engagement action is dependent on GITR and IL-13 expression in vivo. FIG. 6A shows a cohort of GITR^{-/-} mice injected or not with ILC2s from WT mice. After the adoptive transfer, mice were treated with PBS, DTA-1 (1 mg/mice), rmIL-33 (0.5 μg/mice) and fed on HFD for 14 weeks according to the scheme. FIG. 6B shows total weight and FIG. 6C shows fasting blood glucose levels measured every two weeks for 14 weeks. FIG. 6D shows the results of a glucose tolerance test performed after 14 weeks of treatment. FIG. 6E shows a cohort of GITR^{-/-} mice injected with ILC2s from either WT, IL-5^{-/-} or IL-13^{-/-} mice. After the adoptive transfer, mice were treated with PBS, DTA-1 or rm-IL-33 and fed on HFD for 14 weeks according to the scheme. FIG. 6F shows total weight and FIG. 6G shows fasting blood glucose levels measured every two weeks for 14 weeks. FIG. 6H shows results of a glucose tolerance test performed after 14 weeks of treatment.

FIG. 7—GITR engagement induces NF-κB pathway signaling in ILC2s. FIG. 7A shows a heat plot demonstration of modulation of depicted genes in sorted WT ILC2s treated with PBS or DTA-1 (5 μg/mL) for 4 and 24 hours in vitro. FACS-purified ILC2s were quantified by NanoString nCounter technology. Data range from −1 to +1 for the most reduced and most increased gene expression, respectively. FIG. 7B shows a representative histogram of the expression of NF-κB p65 in isolated ILC2s from mice challenged with IL-33 and cultured in vitro for 24 hours with (red) or without (black) DTA-1 (5 μg/mL). The level of isotype-matched stain control is shown as a grey-filled histogram. FIG. 7C shows a corresponding quantification presented as Mean Fluorescence Intensity of NF-κB p65 with or without DTA-1.

FIG. 8—GITR is expressed on human ILC2s and augments Th2-cytokine production. FACS-sorted human blood ILC2s were cultured with recombinant human (rh) IL-2, rhIL-7 and rhIL-33 and plate bound GITR-L-Fc or isotype control, human IgG. FIG. 8A shows representative FACS plots of h-GITR expression after 0, 24 and 48 hours of stimulation on ILC2s and FIG. B shows a corresponding quantitation presented as MFI. FIG. 8C shows a representative FACS plot of h-GITR staining on VAT-ILC2s isolated from healthy subjects and corresponding quantitation. FIG. 8D shows the levels of IL-5, IL-13, GM-CSF, IL-8 and IL-9 in the culture supernatants were measured by Luminex after 24 hours of stimulation. Data is representative of 8 individual blood donors.

FIG. S1—Engagement of GITR induces ILC2 proliferation in bone marrow. Mice were euthanized on 0, 3, 6 and 10 weeks and BM (bone marrow cells) resident ILC2s were quantified at the indicated times (FIG. S1A). Representative FACS plots of BM Lin⁻CD45⁺IL-7R⁺ST2+ ILC2s on week 10 are shown (FIG. S1B).

FIG. S2—High-fat diet induces IL-33 and IL-25 expression in adipose tissue. Mice fed chow or high-fat diet were euthanized on 0, 2, 4, 6, 8, 10 12 and 14 weeks. FIGS. S2A and B show time-kinetic qPCR expression of IL-33 (FIG. S2A) and IL-25 (FIG. S2B) in visceral adipose tissue (VAT) lysates.

FIG. S3—Effects of GITR engagement on physical activity, water and food intake in Rag2^{-/-} mice. A cohort of Rag2^{-/-} mice were fed on High Fat Diet (HFD) and either treated with PBS or DTA-1 (1 mg/mice) by intraperitoneal injection every four days. FIG. S3 shows measured physical activity (FIG. S3A), water (FIG. S3B) and food (FIG. S3C) intake (L: light phase; D: dark phase) after 14 weeks of HFD.

FIG. S4—GITR engagement does not induce Th2-cytokine in ILC2s in lean Rag2^{-/-} mice. FIG. S4A shows a cohort of Rag2^{-/-} mice fed on HFD. Representative flow cytometry plots of ILC2 intracellular staining with IL-5 (left) and IL-13 (right) isotype control antibodies are also shown. FIG. S4B shows a cohort of Rag2^{-/-} mice fed on a chow diet and either treated with PBS or DTA-1 (1 mg/mice) by intraperitoneal injection for 6 consecutive days. Representative flow cytometry plots of intracellular IL-5 (top panel) and IL-13 (bottom panel) in AT ILC2s and corresponding quantitation after 6 days of treatment are shown. Data is presented as frequency of positive IL-5 and IL-13 ILC2s respectively.

FIG. S5—ILC2 gating strategies in human blood and adipose tissue. FIG. S5 shows a gating strategy of Lin⁻CD45⁺CRTH2⁺CD161⁺ILC2 cells in blood (FIG. S5A) and in adipose tissue (FIG. S5B).

### DETAILED DESCRIPTION

Similar to Th2 cells, activated ILC2s can produce significant amounts of IL-5 and IL-13, and can therefore play important roles in regulating metabolic homeostasis in VAT. For example, the production of IL-13 induces differentiation of macrophages towards an anti-inflammatory phenotype, referred to as alternatively activated macrophages (AAMs), whereas IL-5 plays a crucial role in the activation and recruitment of eosinophils, which in turn secrete most of the IL-4 required for the maintenance of AAMs. In addition, ILC2s express MHC class II and costimulatory molecules such as CD80, CD86, ICOS, and we further showed that ILC2s also express ICOS-L.

Glucocorticoid-induced tumor necrosis factor receptor (GITR), also known as TNFRSF18, is a member of the TNFR superfamily which is often found expressed on CD4⁺ and CD8⁺ T lymphocytes. GITR is upregulated in the context of inflammation and acts as an important costimulatory signal in T lymphocyte subpopulations, as studies have shown that engagement of GITR with its ligand (GITRL) in vivo induces T cell expansion and cytokine production. Moreover, GITR was also described as a marker for Treg activation in animal models as its engagement on Tregs led to expansion and paralleled loss of suppressor activity in vitro.

The effects of GITR engagement on ILC2s were evaluated in the context of Type-2 Diabetes Mellitus (T2DM). It was determined, for the first time, that human and mouse ILC2s express the GITR costimulatory receptor. Using a GITR specific agonist and a mouse model of GITR deficiency, it was discovered herein that GITR engagement not only protects against the development of T2DM onset but can also ameliorate established T2DM. It was further demonstrated herein that the protective role of the GITR agonist is IL-13 dependent and that engagement of GITR induces activated ILC2 effector function while increasing the expression of the critical inflammatory modulator NF-κB. Furthermore, GITR is expressed on both naïve and activated ILC2s, highlighting the role of GITR as an immune checkpoint molecule capable of exclusively costimulating activated ILC2s. Accordingly, molecules and compounds that can engage GITR, for example GITR agonists, can be used to prevent or treat T2DM and related disorders such as insulin resistance and obesity.

Presented herein, in some embodiments, are GITR agonists, and compositions (e.g., pharmaceutical compositions) comprising one or more GITR agonists. In some embodiments, a GITR agonist is a binding agent that specifically binds to GITR (e.g., a GITR expressed on the surface of a cell). In some embodiments a GITR agonist presented herein, or a composition comprising a GITR agonist (e.g., a pharmaceutical composition), is used for the treatment, or prevention of diabetes, or a related disorder in a subject.

The term “subject” refers to a mammal. Any suitable mammal can be treated by a method or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. In some embodiments, a mammal is an experimental animal or an animal model of a disease.

In some embodiments a subject is in need of a treatment or composition described herein. In certain embodiments a subject has or is suspected of having diabetes (e.g., diabetes mellitus), or a related disorder. In some embodiments a subject is at risk of having or developing diabetes (e.g., diabetes mellitus), or a related disorder. In certain embodiments a GITR agonist or composition described herein is used to treat a subject having, suspected of having, or at risk of having diabetes (e.g., diabetes mellitus), or a related disorder.

In some embodiments a GITR agonist comprises a binding agent that specifically binds to GITR. In some embodiments a GITR agonist is a binding agent that specifically binds to GITR on the surface of a cell and upon binding, inducing signaling events through the GITR receptor. In some embodiments, a GITR agonist induces cell surface clustering of GITR receptors thereby inducing cytosolic signaling through the GITR receptor. In some embodiments, a GITR agonist is a ligand that specifically binds to a GITR receptor. In some embodiments, a GITR agonist is a GITR ligand that specifically binds to GITR on the surface of a cell and upon binding, inducing signaling events through the GITR receptor. In certain embodiments, a GITR ligand is GITRL, or a functional variant or functional derivative thereof.

In certain embodiments, a binding agent comprises or consists of one or more polypeptides or one or more proteins that bind specifically to at least one antigen (e.g., GITR or a portion thereof). A binding agent often comprises at least one antigen binding portion (i.e. a binding portion). An antigen binding portion of a binding agent is that portion that binds specifically to an antigen. In certain embodiments a binding portion of a binding agent comprises or consists of a single polypeptide (e.g., single chain antibody). In some embodiments, a binding portion of a binding agent comprises or consists of two polypeptides. In some embodiments, a binding portion of a binding agent comprises or consists of 2, 3, 4 or more polypeptides. In some embodiments, a binding agent comprises one or more structural portions (e.g., scaffolds, structural polypeptides, constant regions and/or framework regions). In some embodiments, a binding agent, or binding portion thereof is attached to a substrate (e.g., a polymer, a non-organic material, silicon, a bead, and the like).

A binding agent may comprise one antigen binding portion or multiple antigen binding portions. For example, a binding agent that comprises one binding portion is sometimes referred to as monovalent. A binding agent that comprises two binding portions is sometimes referred as divalent. In some embodiments a binding agent comprises 1, 2, 3, 4, 5, 6, 7, 8, 9, or 10 or more binding portions. In certain embodiments, all of the binding portions of a multivalent binding agent bind to the same antigen. In certain embodiments, all of the binding portions of a multivalent binding agent comprise one or more polypeptide sequences that are at least 90%, at least 95%, at least 99% or 100% identical.

In certain embodiments, two or more binding portions of a binding agent bind to different antigens. Such binding agents are sometimes referred to as bi-specific or multi-specific binding agents (e.g., antibodies). Thus, in certain embodiments a binding agent comprises a first antigen binding portion that specifically binds GITR, or a portion thereof, and a second antigen binding portion that specifically binds another antigen (e.g., a polypeptide that is not GITR, or a portion thereof). A binding agent that specifically binds GITR, in some embodiments, is covalently or non-covalently attached to another binding agent that does not bind specifically to GITR, or a portion thereof. In certain embodiments, a binding agent that specifically binds GITR comprises a second binding agent the specifically binds to another antigen.

In some embodiments a binding agent comprises an antibody, or a portion thereof (e.g., a binding portion thereof). In certain embodiments, a binding agent comprises or consists of a suitable antibody, an antibody fragment and/or an antigen binding portion of an antibody (e.g., a binding fragment, i.e., a binding portion thereof). In some embodiments a binding agent is an antibody (e.g., a monoclonal antibody and/or a recombinant antibody). A binding agent or antibody can be generated, manufactured or produced by a suitable method. In some embodiments a binding agent is monoclonal. In some embodiments a binding agent is a monoclonal antibody derived from a suitable species. Certain non-limiting examples of a binding agent include monoclonal antibodies, chimeric antibodies, antibody binding fragments (e.g., an antigen binding portion of an antibody), a CDR-grafted antibody, a humanized antibody, a human antibody, or portions thereof. Human antibodies can be obtained by any suitable method. For example, human antibodies can be obtained from trans-chromosomal animals engineered to produce fully human antibodies. In certain embodiments, a binding agent is not polyclonal, is not a polyclonal antibody and the term “binding agent” does not refer to polyclonal antibodies.

In some embodiments a binding agent is derived, produced, obtained, isolated, and/or purified from a suitable species. In some embodiments a binding agent is derived, produced, obtained, isolated, and/or purified from a rabbit, goat, horse, cow, rat, mouse, fish, bird, or llama, for example. In some embodiments a binding agent is derived, produced, obtained, isolated, and/or purified from a bird (e.g., a chicken, or a bird egg). In some embodiments a binding agent is derived, produced, obtained, isolated, and/or purified from a plant (e.g., a recombinant binding agent produced by a genetically engineered plant). In some embodiments a binding agent is derived, produced, obtained, isolated, and/or purified from a suitable mammal. In certain embodiments a suitable mammal is a genetically altered mammal (e.g., a trans-chromosomal or transgenic mammal) engineered to produce antibodies comprising human heavy chains and/or human light chains or portions thereof. In some embodiments a binding agent is produced, obtained, isolated, or purified from a prokaryotic or eukaryotic cell (e.g., a recombinant binding agent produced by a genetically engineered cell). In some embodiments a binding agent is produced, obtained, isolated, or purified from a virus (e.g., a recombinant binding agent produced by a genetically engineered virus). A binding agent can be expressed, isolated from and/or purified from a suitable expression system non-limiting examples of which include a suitable bacteria, phage, insect, virus, plant or mammalian expression system. For example, a nucleic acid encoding a binding agent can be introduced into a suitable mammalian cell line that expresses and secretes the binding agent into the cell culture media.

The modifier “monoclonal” is not to be construed as requiring production of a binding agent by any particular method. A monoclonal binding agent can be produced by any suitable method. For example, in certain embodiments, a monoclonal antibody is made by the hybridoma method (e.g., as described by Kohler et al., Nature, 256:495 (1975)), or a variation thereof. In some embodiments a monoclonal binding agent is made by a recombinant DNA method. For example, a monoclonal binding agent can be made by screening a recombinant library using a suitable expression system (e.g., a phage display expression system). In some embodiments a monoclonal binding agent is isolated from a phage library of binding agents, for example by using a technique described in Clackson et al., Nature, 352:624-628 (1991) and/or Marks et al., J. Mol Biol, 222:581-597 (1991), or a variation thereof.

In certain embodiments, a binding agent comprises one or more structural or backbone portions, sometimes referred to as scaffolds. A binding agent may comprise any suitable scaffold, non-limiting examples of which include scaffolds derived from an antibody, a Z domain of Protein A, gamma-B crystalline, ubiquitin, cystatin, Sac7d, a triple helix coiled coil, a lipocalin, an ankyrin repeat motif, an SH3 domain of Fyn, a Kunitz domain of a suitable protease inhibitor, a fibronectin domain, a nucleic acid polymer, the like, portions thereof or combinations thereof. In some embodiments a binding agent does not comprise a scaffold. In certain embodiments, a binding agent comprises one or more structural portions of a mammalian antibody.

In certain embodiments a binding agent comprises one or more constant regions (e.g., constant regions derived from an antibody, e.g., a mammalian antibody). In certain embodiments a binding agent comprises a constant region of an antibody light chain and/or a constant region of an antibody heavy chain. In a mammalian antibody at least two types of immunoglobulin light chains exist which are referred to as lambda (λ) and kappa (κ). A binding agent may comprise any suitable constant region of an antibody, or one or more portions thereof. In some embodiments a binding agent comprises a lambda light chain constant region or a portion thereof. In some embodiments a binding agent comprises a kappa light chain constant region or a portion thereof. In some embodiments a binding agent comprises a polypeptide that is at least 75%, at least 80%, at least 85%, at least 90%, at least 95% or at least 99% identical to a polypeptide sequence of a constant region, or portion thereof, of a light chain of a mammalian antibody. In some embodiments a binding agent comprises a polypeptide that is at least 75%, at least 80%, at least 85%, at least 90%, at least 95% or at least 99% identical to a polypeptide sequence of a constant region of an antibody light chain of a human antibody. In some embodiments a binding agent does not include a light chain constant region.

In certain embodiments a binding agent comprises a constant region of an antibody heavy chain. In mammals, an antibody can have at least five types/classes of Ig heavy chains denoted as IgA, IgD, IgE, IgG, and IgM, which are determined by the presence of distinct heavy chain constant regions, or portion thereof (e.g., CH1, CL, CH2, CH3 domains). A binding agent can include any suitable heavy chain constant region, or portion thereof. In some embodiments a binding agent comprises a heavy chain constant region of an IgG1, IgG2, IgG3 or IgG4, or one or more portions thereof. In some embodiments a binding agent comprises one or more heavy chain constant regions of an IgM, IgD, IgA, or IgE isotype, or a portion thereof. In some embodiments a binding agent comprises a polypeptide that is at least 75%, at least 80%, at least 85%, at least 90%, at least 95%, at least 99% identical, or 100% identical to a polypeptide sequence of a constant region, or portion thereof, of a heavy chain of a mammalian antibody. In some embodiments a binding agent comprises a polypeptide that is at least 75%, at least 80%, at least 85%, at least 90%, at least 95%, at least 99% identical or 100% identical to a polypeptide sequence of a constant region of an antibody heavy chain of a human antibody. In some embodiments a binding agent comprises one or more additions, deletions and/or modification to a constant region. A binding agent is sometimes modified to change the antibody class, or isotype of a binding agent. In some embodiments a binding agent comprises one or more additions, deletions and/or modification (one or more amino acid substitutions, deletions or additions) to modify one or more functions of a binding agent, for example to abolish, enhance or decrease serum half-life, Fc receptor binding, complement binding (e.g., C1q binding), glycosylation, sialylation, cellular toxicity, antibody-dependent cell-mediated phagocytosis (ADCP), antibody dependent cellular cytotoxicity (ADCC), and the like. In some embodiments a binding agent does not include one or more portions of a heavy chain constant region or light chain constant region. In some embodiments a binding agent does not include a heavy chain constant region.

In some embodiments a binding agent comprises or consists of one or more variable regions of an antibody, or a portion thereof. In some embodiments a binding agent comprises one or more light chain variable regions, or a portion thereof. In some embodiments a binding agent comprises one or more heavy chain variable regions, or a portion thereof. In certain embodiments a binding agent comprises at least one light chain variable region and at least one heavy chain variable region. A light chain variable region and heavy chain variable region can be on the same or different polypeptides. In certain embodiments, an antigen binding portion of a binding agent consists of one or more heavy chain variable regions. In certain embodiments, an antigen binding portion of a binding agent consists of one or more light chain variable regions. In certain embodiments, an antigen binding portion of a binding agent consists of one or more light chain variable regions and one or more heavy chain variable regions.

In some embodiments a binding agent comprises or consists of a Fab, Fab′, F(ab′)2, Fv fragment, single-chain Fv (scFv), diabody (Dab), synbody, the like and/or a combination or portion thereof. In some embodiments a binding agent is a Fab, Fab′, F(ab′)2, Fv fragment, single-chain Fv (scFv), diabody (Dab), synbody, the like and/or a combination, or portion thereof (see, e.g., U.S. Pat. Nos. 6,099,842 and 5,990,296). In some embodiments a binding agent comprises a single-chain polypeptide comprising one or more antigen binding portions. For example, a single-chain binding agent can be constructed by joining a heavy chain variable region, or antigen binding portion thereof, with a light chain variable region, or antigen binding portion thereof, with a linker (e.g., an amino acid, a polypeptide linker) using recombinant molecular biology processes. Such single chain binding agents often exhibit specificities and affinities for an antigen similar to a parent two-chain monoclonal binding agent. Binding agents often comprise engineered regions such as CDR-grafted or humanized portions. In certain embodiments a binding agent is an intact two-chain immunoglobulin, and in other embodiments a binding agent is a Fab monomer or a Fab dimer.

The term “percent identical” or “percent identity” refers to sequence identity between two amino acid sequences. Identity can be determined by comparing a position in each sequence which may be aligned for purposes of comparison. When an equivalent position in the compared sequences is occupied by the same amino acid, then the molecules are identical at that position. When the equivalent site is occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Methods of generating chimeric, humanized and/or optimized antibodies or binding agents, for example by modifying, substituting or deleting framework regions, or portions thereof, are known. Non-limiting examples of CDR grafting are described, e.g., in U.S. Pat. Nos. 6,180,370, 6,054,297, 5,693,762, 5,859,205, 5,693,761, 5,565,332, 5,585,089, and 5,530, 101, and in Jones et al, Nature, 321:522-525 (1986); Verhoeyen et al., Science, 239:1534-1536 (1988), and Winter, FEBS Letts., 430:92-94 (1998). Additional non-limiting examples of generating chimeric, grafted and/or humanized binding agents include U.S. Pat. Nos. 5,530,101; 5,707,622; 5,994,524; 6,245,894; Queen et al., (1988) PNAS 86:10029-10033; Riechmann et al., Nature (1988) 332:323-327; Antibody Engineering: Methods and Protocols, Vol. 248 of Methods in molecular biology, edited by Benny K. C. Lo, Springer Science & Business Media, (2004); and Antibody Engineering, Vol. 1, Roland E. Kontermann, Stefan Dubël, Edition 2, Publisher Springer Science & Business Media, (2010). In some embodiments a binding agent can be humanized by exchanging one or more framework regions, or portions thereof (e.g., one or more amino acids), with one or more framework regions, or portions thereof from a human antibody. In certain embodiments, an antibody or binding agent can be humanized or grafted by transferring one or more CDRs (e.g., 1, 2, 3, 4, 5 or all 6 CDRs) from a donor binding agent (e.g., a mouse monoclonal antibody; e.g., DTA-1) to an acceptor binding agent (e.g., a human antibody) while retaining the binding specificity of the donor binding agent. In certain embodiments, the process of making a chimeric, grafted or humanized binding agent comprises making one or more amino acid substitutions, additions or deletions in a constant region or framework region of a binding agent. In certain embodiments, techniques such as “reshaping”, “hyperchimerization,” or “veneering/resurfacing” can be used to produce humanized binding agents. (e.g., see Vaswami et al, Annals of Allergy, Asthma, & Immunol. 81:105 (1998); Roguska et al., Prot. Engin., 9:895-904 (1996); and U.S. Pat. No. 6,072,035). In some aspects, a binding agent is modified by a method discussed above, or by another suitable method, to reduce immunogenicity (e.g., see Gilliland et al., J. Immunol, 62(6):3663-71 (1999)).

In certain embodiments, an amino acid sequence of a binding agent is modified to optimize binding affinity for a GITR, species cross-reactivity, solubility and/or function (e.g., agonist activity). In some embodiments the specific combination of CDRs of DTA-1 can be optimized for binding to GITR, and/or to optimize agonist activity. In certain embodiments a GITR agonist is modified to include certain amino acid additions, substitutions, or deletions designed or intended, for example, to reduce susceptibility of a GITR agonist to proteolysis, reduce susceptibility of a GITR agonist to oxidation, increase serum half-life and/or confer or modify other physicochemical, pharmacokinetic or functional properties of a GITR agonist.

In some embodiments a GITR agonist specifically binds to a mammalian GITR, or portion thereof. In some embodiments a GITR agonist specifically binds to an extracellular domain or extracellular regions of a mammalian GITR, or a portion thereof. In certain aspects, a GITR agonist specifically binds to a wild-type GITR produced by a cell of an unaltered (non-genetically modified) mammal found in nature. In certain aspects a GITR agonist specifically binds to a naturally occurring GITR variant. In certain aspects a GITR agonist specifically binds to a GITR comprising one or more amino acid substitutions, additions or deletions. In certain embodiments a GITR agonist specifically binds to a GITR produced and/or expressed on the surface of a cell of a human, non-human primate, dog, cat, or rodent (e.g., a mouse or rat). In certain embodiments, a GITR agonist specifically binds to an extracellular domain of human GITR.

The term “specifically binds” refers to a GITR agonist (e.g., a GITR ligand or binding agent) that binds a target peptide in preference to binding other molecules or other peptides as determined by, for example, as determined by a suitable in vitro assay (e.g., an Elisa, Immunoblot, Flow cytometry, and the like). A specific binding interaction discriminates over non-specific binding interactions by about 2-fold or more, often about 10-fold or more, and sometimes about 100-fold or more, 1000-fold or more, 10,000-fold or more, 100,000-fold or more, or 1,000,000-fold or more.

In some embodiments a GITR agonist that specifically binds to GITR, or a portion thereof, is a GITR agonist that binds GITR, or a portion thereof (e.g., an extracellular domain of GITR), with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM.

In certain embodiments, a GITR agonist comprises one or more functional characteristics. Accordingly, a GITR agonist can be described structurally and functionally (e.g., by what it does, or by what it is capable of doing). For example, GITR agonists disclosed herein are agonistic and can therefore induce or promote signaling through GITR (e.g., Nf-kB activation, or other cell signaling events). GITR agonists disclosed herein can bind specifically to an extracellular portion of GITR, for example, an extracellular portion of GITR present on the surface of an ILC2 cell. An ILC2 cell that expresses GITR on its cell surface can be any suitable mammalian ILC2 cell (e.g., a human or mouse ILC2).

In some embodiments, a GITR agonist comprises DTA-1, or an binding fragment thereof. In some embodiments, a GITR agonist is a humanized version of DTA-1 which can contain human constant regions and/or human framework regions. In some embodiments a GITR agonist is an antibody or antigen binding portion thereof that comprises the three CDRs of the heavy chain and three CDRs of the light chain of DTA-1. In some embodiments, a GITR agonist comprises a chimeric version of DTA-1 wherein the constant regions are replaced with human constant regions.

In some embodiments, a GITR agonist comprises MK-4166, or an binding fragment thereof, as disclosed in Sukumar et al., (2017) Cancer Res. 77(16):4378-4388, which is incorporated herein by reference. In some embodiments, a GITR agonists is a humanized version of MK-4166.

In some embodiments, a GITR agonist comprises or consists of an agonist monoclonal antibody, agonists binding agent or agonist ligand that specifically binds to human GITR selected from an antibody or binding agent disclosed in U.S. Pat. Nos. 9,701,751, 9,241,992, 7,812,135, U.S. patent Ser. No. 10/155,818, U.S. Patent Application Publication No. 2018/0244752, U.S. Patent Application Publication No. 2007/0098719, U.S. Patent Application Publication No. 2005/0014224, U.S. Patent Application Publication No. 2015/0064204, U.S. Patent Application Publication No. 2017/0260282, U.S. Patent Application Publication No 20190010241 or International Patent Application Publication No. WO05007190, all of which are incorporated herein by reference. In some embodiments, a GITR agonist is an agonist anti-GITR antibody or a GITR agonist disclosed in U.S. Pat. No. 9,499,627, which is incorporated herein by reference.

In some embodiments, presented herein is a composition or pharmaceutical composition comprising one or more GITR agonists (e.g., binding agents or GITR ligands) that binds specifically to GITR, or a portion thereof (e.g., an extracellular domain of GITR, or a portion thereof).

A pharmaceutical composition can be formulated for a suitable route of administration. In some embodiments a pharmaceutical composition is formulated for subcutaneous (s.c.), intradermal, intramuscular, intraperitoneal and/or intravenous (i.v.) administration. In certain embodiments, a pharmaceutical composition can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants (Remington's Pharmaceutical Sciences, 18th Ed., A. R. Gennaro, ed., Mack Publishing Company (1995)).

In certain embodiments, a pharmaceutical composition comprises a suitable excipient, non-limiting example of which include anti-adherents (e.g., magnesium stearate), a binder, fillers, monosaccharides, disaccharides, other carbohydrates (e.g., glucose, mannose or dextrins), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweeteners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington's Pharmaceutical Sciences, 18th Ed., A. R. Gennaro, ed., Mack Publishing Company (1995). The term “binder” as used herein refers to a compound or ingredient that helps keeps a pharmaceutical mixture combined. Suitable binders for making pharmaceutical formulations and are often used in the preparation of pharmaceutical tablets, capsules and granules are known to those skilled in the art. For clarification, the term “binding agent” as used herein does not refer to a “binder” that is used in certain pharmaceutical formulations. Although a pharmaceutical composition, in certain embodiments, may comprise a binding agent that specifically binds GITR as well as a binder.

In some embodiments a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agent includes those having a sulfhydryl group such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments a pharmaceutical composition comprises one or more suitable preservatives. Non limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments a pharmaceutical composition is free of preservatives.

In some embodiments a composition, pharmaceutical composition or GITR agonist is substantially free of blood, or a blood product contaminant (e.g., blood cells, platelets, polypeptides, minerals, blood borne compounds or chemicals, and the like). In some embodiments a composition, pharmaceutical composition or GITR agonist is substantially free of serum and serum contaminants (e.g., serum proteins, serum lipids, serum carbohydrates, serum antigens and the like). In some embodiments a composition, pharmaceutical composition or GITR agonist is substantially free of a pathogen (e.g., a virus, parasite or bacteria). In some embodiments a composition, pharmaceutical composition or GITR agonist is substantially free of endotoxin. In some embodiments a composition, pharmaceutical composition or GITR agonist is sterile. In certain embodiments, a composition or pharmaceutical composition comprises a GITR agonist that specifically binds an extracellular domain of GITR and a diluent (e.g., phosphate buffered saline). In certain embodiments, a composition or pharmaceutical composition comprises a GITR agonist that specifically binds an extracellular domain of GITR and an excipient, (e.g., sodium citrate dehydrate, or polyoxyethylene-sorbitan-20 mono-oleate (polysorbate 80)).

The pharmaceutical compositions described herein may be configured for administration to a subject in any suitable form and/or amount according to the therapy in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents. In some embodiments a pharmaceutical composition suitable for parenteral administration may contain one or more excipients. In some embodiments a pharmaceutical composition is lyophilized to a dry powder form. In some embodiments a pharmaceutical composition is lyophilized to a dry powder form, which is suitable for reconstitution with a suitable pharmaceutical solvent (e.g., water, saline, an isotonic buffer solution (e.g., PBS), and the like). In certain embodiments, reconstituted forms of a lyophilized pharmaceutical composition are suitable for parenteral administration (e.g., intravenous administration) to a mammal.

In some embodiments a pharmaceutical compositions described herein may be configured for topical administration and may include one or more of a binding and/or lubricating agent, polymeric glycols, gelatins, cocoa-butter or other suitable waxes or fats. In some embodiments a pharmaceutical composition described herein is incorporated into a topical formulation containing a topical carrier that is generally suited to topical drug administration and comprising any suitable material known to those skilled in the art. In certain embodiments, a topical formulation of a pharmaceutical composition is formulated for administration of a GITR agonist from a topical patch.

In certain embodiments, an optimal pharmaceutical composition will be determined by one skilled in the art depending upon, for example, the intended route of administration, delivery format and desired dosage (see e.g., Remington's Pharmaceutical Sciences, supra). In certain embodiments, such compositions may influence the physical state, stability, rate of in vivo release and rate of in vivo clearance of the antibodies of the invention.

In some embodiments a composition, pharmaceutical composition or GITR agonist described herein is used to treat a subject having or suspected of having diabetes or a related disorder. In certain embodiments, a GITR agonist or pharmaceutical composition described herein is used in treating diabetes or a related disorder in a subject, wherein the GITR agonist specifically binds to an extracellular domain of human GITR. Is some embodiments, presented herein is a method of treating a subject having or suspected of having diabetes or a related disorder. In certain embodiments, a method of treating a subject having or suspected of having diabetes or a related disorder comprises administering a therapeutically effective amount of a composition, pharmaceutical composition or GITR agonist described herein to the subject. In certain embodiments, a method of treatment comprises contacting one or more ICL2 cells of a subject with a therapeutically effective amount of a composition, pharmaceutical composition or GITR agonist described herein. In certain embodiments, a method of treatment comprises contacting one or more ICL2 cells of a subject with a therapeutically effective amount of a GITR agonist that specifically binds to an extracellular portion of human GITR, or functional variant thereof. An ILC2 cell is often a cell that expresses an extracellular portion of GITR on its cell surface. An ILC2 cell of a subject may be found inside a subject (e.g., in vivo) or outside the subject (e.g., in vitro or ex vivo).

A composition, pharmaceutical composition or GITR agonist disclosed herein can be used to treat diabetes or a related disorder. Non-limiting examples of diabetes and related disorders that can be treated by a method herein includes Type 1 diabetes, Immune mediated Type I diabetes, Idiopathic Type I diabetes, Type-2 Diabetes, Type-2 Diabetes Mellitus, Type-2 Diabetes as a result of genetic defects of ß-cell function, Type-2 Diabetes as a result of genetic defects in insulin action (e.g., Type A insulin resistance, Leprechaunism, Rabson-Mendenhall syndrome, Lipoatrophic diabetes), diseases of the exocrine pancrease (e.g., Pancreatitis, Trauma/pancreatectomy, Neoplasia, Cystic fibrosis, Hemochromatosis, Fibrocalculous pancreatopathy), Endocrinopathies (e.g., Acromegaly, Cushing's syndrome, Glucagonoma, Pheochromocytoma, Hyperthyroidism, Somatostatinoma, Aldosteronoma), Drug- or chemical-induced diabetes (e.g., Vacor, Pentamidine, Nicotinic acid, Glucocorticoids, Thyroid hormone, Diazoxide, ß-adrenergic agonists, Thiazides, Dilantin, ∝-Interferon), rare type of immune-mediated diabetes (e.g., “Stiff-man” syndrome and anti-insulin receptor antibodies), obesity (e.g., chronic obesity, central obesity), insulin resistance, hyperlipidemia, and hypertension, and gestational diabetes mellitus. Additional non-limiting examples of diabetes-related disorders that can be prevented or treated by a method disclosed herein include glucose intolerance, dyslipidemia with elevated triglycerides, Low HDL-cholesterol, Microalbuminuria, redominance of small dense LDL-cholesterol particles, Hypertension, Endothelial dysfunction, Oxidative stress, fatty liver disease (NASH), and gout. In some embodiments, a GITR agonists, or a composition (e.g., a pharmaceutical composition) disclosed herein can be used to treat Type-2 diabetes mellitus.

Any suitable method of administering a composition, pharmaceutical composition or GITR agonist to a subject can be used. The exact formulation and route of administration for a composition for use according to the methods of the invention described herein can be chosen by the individual physician in view of the patient's condition. See, e.g., Fingl et al. 1975, in “The Pharmacological Basis of Therapeutics,” Ch. 1, p. 1; which is incorporated herein by reference in its entirety. Any suitable route of administration can be used for administration of a pharmaceutical composition or a GITR agonist described herein. Non-limiting examples of routes of administration include topical or local (e.g., transdermally or cutaneously, (e.g., on the skin or epidermis)), in or on the eye, intranasally, transmucosally, in the ear, inside the ear (e.g., behind the ear drum), enteral (e.g., delivered through the gastrointestinal tract, e.g., orally (e.g., as a tablet, capsule, granule, liquid, emulsification, lozenge, or combination thereof)), sublingual, by gastric feeding tube, rectally, by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a composition herein is provided to a subject. A composition that is provided to a subject is sometimes provided to a subject for self-administration or for administration to a subject by another (e.g., a non-medical professional). For example, a composition described herein can be provided as an instruction written by a medical practitioner that authorizes a patient to be provided a composition or treatment described herein (e.g., a prescription). In another example, a composition can be provided to a subject where the subject self-administers a composition orally, intravenously or by way of an inhaler, for example.

Alternately, one can administer compositions for use according to the methods of the invention in a local rather than systemic manner, for example, via direct application to the skin, mucous membrane or region of interest for treating, including using a depot or sustained release formulation.

In some embodiments a pharmaceutical composition comprising a GITR agonist can be administered alone (e.g., as a single active ingredient (AI or e.g., as a single active pharmaceutical ingredient (API)). In other embodiments, a pharmaceutical composition comprising a GITR agonist can be administered in combination with one or more additional AIs/APIs, for example, as two separate compositions or as a single composition where the one or more additional AIs/APIs are mixed or formulated together with the GITR agonist in a pharmaceutical composition.

A pharmaceutical composition can be manufactured by any suitable manner, including, e.g., by means of conventional mixing, dissolving, granulating, dragee-making, levigating, emulsifying, encapsulating, entrapping or tableting processes.

In some embodiments a pharmaceutical composition comprising a GITR agonist is administered at a suitable frequency or interval as needed to obtain an effective therapeutic outcome. An effective therapeutic outcome can be determined by monitoring one or more clinical aspects of diabetes or a related disorder. Accordingly, in certain embodiments, a decrease in one or more clinical markers or clinical symptoms of diabetes or a related disorder is considered an effective therapeutic outcome. In some embodiments, a pharmaceutical composition comprising a GITR agonist can be administered hourly, once a day, twice a day, three times a day, four times a day, five times a day, and/or at regular intervals, for example, every day, every other day, three times a week, weekly, every other week, once a month and/or simply at a frequency or interval as needed or recommended by a medical professional.

In some embodiments, an amount of a GITR agonist in a composition is an amount needed to obtain an effective therapeutic outcome. In certain embodiments, the amount of a GITR agonist in a composition (e.g., a pharmaceutical composition) is an amount sufficient to prevent, treat, reduce the severity of, delay the onset of, and/or alleviate a symptom of a diabetes or a related disorder, as contemplated herein.

A “therapeutically effective amount” means an amount sufficient to obtain an effective therapeutic outcome and/or an amount necessary or sufficient to prevent, treat, reduce the severity of, delay the onset of, and/or alleviate a symptom of diabetes or a related disorder. In certain embodiments, a “therapeutically effective amount” means an amount necessary or sufficient to reduce, decrease or normalize a disease indicator or marker of diabetes or a related disorder (e.g., blood or urine markers, e.g., levels, or amounts of glucose, insulin, glucagon, A1c (HbA1c), proinsulin, C-peptide, C-reactive protein, cytokines, hormones, byproducts thereof and combinations thereof. Determination of a therapeutically effective amount is well within the capability of those skilled in the art, especially in light of the detailed disclosure provided herein.

In some embodiments, an amount of a GITR agonist (e.g., the amount of a GITR agonist in a composition) is an amount that is at least a therapeutically effective amount and an amount low enough to minimize unwanted adverse reactions. The exact amount of a GITR agonist or combinations of active agents required will vary from subject to subject, depending on age, weight, and general condition of a subject, the severity of the condition being treated, and the particular combination of drugs administered. Thus, it is not always possible to specify an exact therapeutically effective amount to treat diabetes or a related disorder in a diverse group of subjects. As is well known, the specific dosage for a given patient under specific conditions and for a specific disease will routinely vary, but determination of the optimum amount in each case can readily be accomplished by simple routine procedures. Thus, a therapeutically effective amount of a GITR agonist used to treat diabetes or a related disorder may be determined by one of ordinary skill in the art using routine experimentation.

In certain embodiments, an amount of a GITR agonist in a composition is administered at a suitable therapeutically effective amount or a dose (e.g., at a suitable volume and concentration, which sometimes depends, in part, on a particular route of administration). Within certain embodiments, a GITR agonist (e.g., a GITR agonist in a composition) can be administered at a dose from about 0.01 mg/kg (e.g., per kg body weight of a subject) to 500 mg/kg, 0.1 mg/kg to 500 mg/kg, 0.1 mg/kg to 400 mg/kg, 0.1 mg/kg to 300 mg/kg, 0.1 mg/kg to 200 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg or 0.1 mg/kg to 1 mg/kg. In some aspects the amount of a GITR agonist can be about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg. In some embodiments a therapeutically effective amount of a GITR agonist is between about 0.1 mg/kg to 500 mg/kg, or between about 1 mg/kg and about 300 mg/kg. Volumes suitable for intravenous administration are well known.

A pharmaceutical composition comprising an amount or dose of a GITR agonist can, if desired, be provided in a kit, pack or dispensing device, which can contain one or more doses of a GITR agonist. The pack can for example comprise metal or plastic foil, such as a blister pack. The pack or dispenser device can be accompanied by instructions for administration. The pack or dispenser can also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert.

In some embodiments a kit or pack comprises an amount of a GITR agonist sufficient to treat a patient for 1 day to 1 year, 1 day to 180 days, 1 day to 120 days, 1 day to 90 days, 1 day to 60 days, 1 day to 30 days, or any day or number of days there between, 1-4 hours, 1-12 hours, or 1-24 hours.

A kit optionally includes a product label or packaging inserts including a description of the components or instructions for use in vitro, in vivo, or ex vivo, of the components therein. Exemplary instructions include instructions for a diagnostic method, treatment protocol or therapeutic regimen. In certain embodiments, a kit comprises packaging material, which refers to a physical structure housing components of the kit. The packaging material can maintain the components sterilely, and can be made of material commonly used for such purposes (e.g., paper, corrugated fiber, glass, plastic, foil, ampules, vials, tubes, etc.). Product labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards. Product labels or inserts can include identifying information of one or more components therein, dose amounts, clinical pharmacology of the active ingredient(s) including mechanism of action, pharmacokinetics (PK) and pharmacodynamics (PD). Product labels or inserts can include information identifying manufacturer information, lot numbers, manufacturer location, date, information on an indicated condition, disorder, disease or symptom for which a kit component may be used. Product labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include labels or instructions for practicing any of the methods and uses of the invention described herein. Product labels or inserts can include information on potential adverse side effects and/or warnings.

In certain embodiments, a kit comprises one or more controls having a known amount of a GITR agonist. In some embodiments, a kit comprises cells expressing GITR or cells expressing a GITR agonist. The cells in the kit can be maintained under appropriate storage conditions until the cells are ready to be used.

### EXAMPLES

### Example 1—Material and Methods

I. Mice

GITR deficient mice were obtained from Dr. Tania Watts (University of Toronto, Toronto, Canada) and Dr. Carlo Riccardi (University of Perugia, Perugia, Italy). C57BL/6J, Ob/Ob (B6.Cg-Lep^{ob}/J), RAG2 deficient (C.B6(Cg)-Rag2^{tm1.1Cgn}/J), IL-13 deficient (C.129P2-Ill3^{tm1.1Anjm}) and IL-5 deficient (C57BL/6-Il5^{tm1Kopf}/J) mice were purchased from the Jackson Laboratory (Bar Harbor, Me.). Four to eight week old aged and sexed matched mice were used in the studies.

### Diet-Induced Obesity and In Vivo Treatments

When indicated, mice were fed a high fat diet (HFD, Rodent diet with 60 kcal % Fat, D12492i) from Research Diets Inc. (New Brunswick, N.J.) for the indicated times, as described before (Trompette et al., 2014). All other mice were fed a standard chow diet. For in vivo experiments investigating the effect of GITR engagement, GITR agonist DTA-1 (1 mg/mouse, BioXCell, West Lebanon, N.H., BE0063), carrier free recombinant mouse (rm)-IL-33 (0.5 μg/mouse) or PBS was administered intraperitoneally every 4 days from the indicated start of treatment until termination of the experiment.

### In Vivo Metabolic Phenotyping

To measure weight and fasting blood glucose levels, mice were fasted overnight (˜14-16 h), weighed and glucose values were measured using a glucometer (Contour® Next EZ, Bayer, Leverkusen, Germany) collecting a drop of blood every two weeks. For intraperitoneal glucose tolerance tests (ip-GTT), mice were fasted overnight (˜16 hours), weighed and injected with 2 g/kg 20% D-glucose (Sigma Aldrich) solution intraperitoneally. Blood glucose values were measured for each mouse by collecting a drop of blood before injection and at 20, 40, 60, 90, 120, 150, 180, 210 and 240 min post-injection. For insulin tolerance tests (ITT), mice were fasted for 5 hours, weighed and injected with 0.5 U/kg human insulin (Novolin®, Novo Nordisk®, Bagsværd, Denmark) diluted in Sodium Chloride Solution 0.9% w/v (Azer Scientific, Morgantown, Pa.) solution intraperitoneally. Blood glucose values were measured for each mouse by collecting a drop of blood before injection and at 20, 40, 60, 90, 120, 150, 180, 210 and 240 min post injection. For both glucose and insulin tolerance tests, the data were analyzed by quantifying the area under the curve (AUC) for each group of mice. When indicated, blood was collected by cardiac puncture and plasma insulin levels were measured using the ultra-sensitive mouse insulin ELISA Kit (Crystal Chem High Performance Assays). Metabolic analysis of whole animals were performed using PhenoMaster/LabMaster home cages following the manufacturer's instructions (TSE Systems). Briefly, at the indicated time after onset of treatment, mice were singly housed and measures were taken every 27 minutes for 5 days. Measures included oxygen consumption and carbon dioxide output, as variations in oxygen consumption and energy expenditure (heat) over time were calculated and normalized to body mass.

### Murine ILC2 Isolation and In Vitro Stimulation

Murine lung and human peripheral ILC2s were isolated to >95% purity using the FACS Aria III cell sorter. For in vivo stimulation of murine lung ILC2s, carrier free rm-IL-33 (Biolegend, San Diego, Calif., 0.5 μg/mouse in 50 μL) or PBS (50 μL) was administered intranasally to mice on three consecutive days. On day 4, murine ILC2s were isolated based on the lack of expression of classical lineage markers (CD3e, CD45R, Gr-1, CD11c, CD11b, Ter119, TCRgd and FCeRI) and expression of CD45, ST2, and CD117. Isolated ILC2s were stimulated (5×10⁴/mL) with rm-IL-2 (10 ng/mL) and rm-IL-7 (10 ng/mL) for 48 hours at 37° C. in presence or absence of GITR agonist DTA-1 (5 μg/mL) from BioXCell, West Lebanon, N.H., BE0063). For adoptive transfer experiments, 2.5×10⁵ purified ILC2s were adoptively transferred intravenously in 200 μL PBS into the recipients at the start of the indicated treatment.

### Human ILC2 Isolation and In Vitro Stimulation

For human ILC2s from blood, peripheral blood mononuclear cells (PBMCs) were first isolated from human fresh blood by diluting the blood 1:1 in PBS and adding to SepMate™-50 separation tubes (STEMCELL Technologies Inc, Vancouver, Canada) prefilled with 15 mL Lymphoprep™ each (Axis-Shield, Oslo, Norway) and centrifugation at 1200 g for 15 minutes. For human ILC2s from adipose tissue, adipose tissue samples were digested in collagenase IV (MP Biomedicals, LLC) at 37° C. for one hour and then processed on a 70 μm nylon cell strainer (Falcon®) into a single cell suspension. Human ILC2s were isolated based on the lack of expression of classical lineage markers (CD3, CD14, CD16, CD19, CD20, CD56, CD235a, CD1a, CD123) and expression of CD45, CRTH2 and CD161. Purified human ILC2s were stimulated (5×10⁴/mL) with recombinant human (rh)-IL-2 (20 ng/mL), rh-IL-7 (20 ng/mL) and rh-IL-33 (20 ng/mL) for the indicated times at 37° C. in the presence or absence of plate-bound GITR-L-Fc (Hu et al., 2008).

II. Supernatant Cytokine Measurement

Human IL-5 ELISA MAX™ Deluxe was purchased from BioLegend, Ready-SET-Go!®. ELISA for human IL-13, mouse IL-5 and IL-13 were purchased from ThermoFisher Scientific and the level of cytokines were measured according to the manufacturer's instructions. Other cytokines were measured by multiplexed fluorescent bead-based immunoassay detection (MILLIPLEX® MAP system, Millipore Corporation, Missouri, USA) according to the manufacturer's instructions, using a combination of 32-plex (MCYTMAG70KPX32) and 41-plex (HCYTMAG-60K-PX41) Millipore Human Cytokine panel kits. For each assay, the curve was performed using various concentrations of the cytokine standards assayed in the same manner and analyzed using MasterPlex2012 software (Hitachi Solutions America, Ltd.), as described by our group before (Galle-Treger, L., et. al. (2016) Nature communications 7:13202; Rigas, D., et al. (2017) The Journal of allergy and clinical immunology 139:1468-1477).

### Tissue Preparation and Flow Cytometry

Lung, perigonadal adipose tissue used as representative VAT and BM were collected at the indicated times after transcardial perfusion to clear organs of red blood cells. Lungs and VAT were processed to single cell suspensions as previously described (Kerzerho, J., et al. (2013) The Journal of Allergy and Clinical Immunology 131:1048-1057), and BM cells were collected by flushing bones with PBS. Stained cells were analyzed on FACSCanto II and/or FACSARIA III systems (Becton Dickinson) and the data were analyzed with FlowJo version 10 software (TreeStar, Ashland, Oreg.). The following mouse antibodies were used: biotinylated anti-mouse lineage CD3e (145-2C11), CD45R (RA3-3B2), Gr-1 (RB6-8C5), CD11c (N418), CD11b (M1/70), Ter119 (TER-119), FceRIa (MAR-1) (BioLegend) and TCR-gd (eBioGL3) (eBioscience), Streptavidin-FITC, PE-Cy7 anti-mouse CD127 (A7R34), APCCy7 anti-mouse CD45 (30-F11), APCeFluor780 anti-mouse CD11c (N418), PECy7 anti-mouse CD45 (13/2.3), PE anti-mouse F4/80 (BM8), FITC anti-mouse CD206 (C068C2), APC anti-mouse CD301 (LOM-14) were purchased from BioLegend. AlexaFluor647 anti-mouse CD357 (GITR) (DTA-1) and isotype control AlexaFluor647 anti-mouse Rat IgG2b were purchased from BD Bioscience. PerCP-eFluor710 anti-mouse ST2 (RMST2-2) and eFluor450 anti-mouse CD11b (M1/70) were purchased from eBioscience. Intranuclear staining was performed using the Foxp3 Transcription Factor Staining Kit (ThermoFisher Scientific), according to the manufacturer's instructions. PE anti-human/mouse RelA NFκB p65 (IC5078P) was purchased from R&D Systems. Intracellular staining was performed using the BD Cytofix/Cytoperm kit (BD Bioscience, San Jose, Calif.), according to the manufacturer's instructions. PE anti-mouse/human IL-5 (Biolegend) and Alexa647 anti-mouse IL-13 (eBioscience) were used. The following human antibodies were used: biotinylated anti-human lineage (CD3, CD14, CD16, CD19, CD20, CD56, CD235a, CD1a, CD123), APCCy7 anti-human CD45, PerCPCy5.5 anti-human CRTH2, PE anti-human CD161, PECy7 anti-human CD127, APC anti-human GITR and corresponding isotype control.

Gene Expression Analysis Using NanoString nCounter Technology

Freshly isolated ILC2s after 3 i.n. rm-IL33 administration were stimulated (5×10⁴/mL) with rm-IL-2 (10 ng/mL) and rm-IL-7 (10 ng/mL) for the indicated times at 37° C. in presence or absence of GITR agonist DTA-1 (5 μg/mL). Total RNA was isolated using MicroRNAeasy (Qiagen, Valencia, Calif.) and the gene expression in ILC2s over time of DTA-1 stimulation was analyzed with NanoString nCounter technology (Seattle, Wash.) and ranged from −1 for the max decrease to +1 for the max increase, as described before (Galle-Treger et al., 2016; Rigas et al., 2017). Heat plots were generated with R statistical software.

### Histologic Analysis

Samples were collected and fixed for histology with 4% paraformaldehyde in PBS. After fixation, samples were embedded in paraffin, cut into 4 mm sections, and stained with hematoxylin and eosin (H&E). Histology pictures were acquired using a Leica DME microscope and Leica ICC50HD camera (Leica, Wetzlar, Germany) and analyzed using Leica LAS EZ software. Adipocyte size quantitation was performed using the Adiposoft plugin of ImageJ (NIH, Maryland, USA).

III. Statistical Analysis

Experiments were repeated at least three times (n=4-6 each) and data are shown as the representative of 3 independent experiments. A student t-test for unpaired data was used for comparisons between each group using Prism Software (GraphPad Software Inc.). The degree of significance were indicated as: *p<0.05, **p<0.01, ***p<0.001.

### Example 2—Results

### GITR is Expressed on ILC2s and Induces Th2-Cytokines

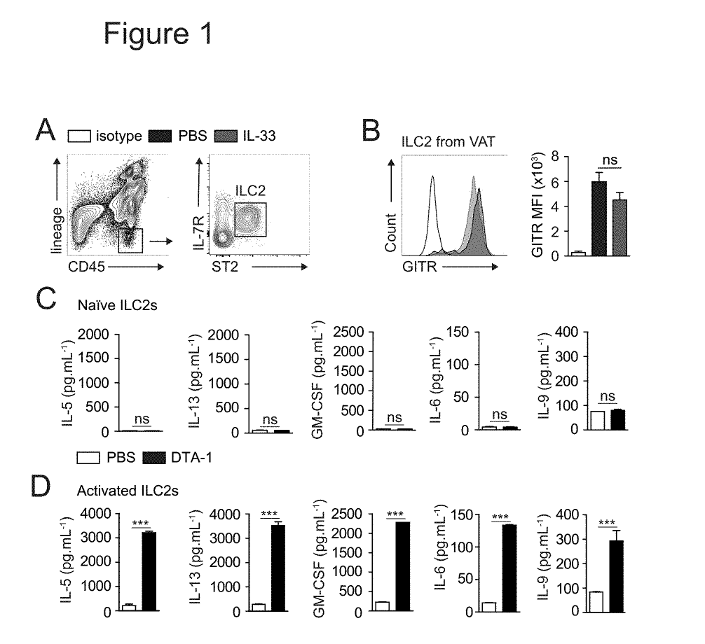

It was first determined if GITR is expressed on naïve and IL-33-activated ILC2s. Mice received intraperitoneal IL-33 or PBS on 3 consecutive days. On day four, ILC2s from visceral adipose tissue (VAT) were analyzed by flow cytometry and gated as lineage⁻CD45⁺IL-7R⁺ and ST2⁺ (FIG. 1A). Further analysis of the cell-surface phenotype of ILC2s showed that both naïve and IL-33-induced ILC2s had high expression of GITR in comparison to the isotype control, although there were no differences in expression between naïve and activated cells (FIG. 1B). To assess the effect of GITR engagement on ILC2 activation, we measured the levels of cytokine secretion in presence or absence of specific GITR agonist, DTA-1. Freshly isolated VAT-derived ILC2s were stimulated in vitro for 48 hours with or without specific DTA-1 (naïve ILC2s, FIG. 1C). As a comparison, freshly isolated VAT-derived ILC2s were stimulated in presence of recombinant mouse (rm)IL-33 for 48 hours with or without DTA-1 (activated ILC2s, FIG. 1D). Cytokine secretion was then measured on the cell culture supernatants. Cytokine production by naïve ILC2s was not affected by DTA-1 treatment (FIG. 1C). In contrast, when activated with rmIL-33, GITR engagement induced secretion of high amounts of IL-5, IL-13, GM-CSF, IL-6 and IL-9 compared to controls (FIG. 1D). Taken together, these results show that even though GITR is expressed on both naïve and activated ILC2s, the induction of Th2-cytokine secretion after GITR engagement requires ILC2s to be activated, suggesting a co-stimulatory role of the GITR receptor in ILC2s.

### Engagement of GITR Protects Against Obesity-Induced Metabolic Disturbances

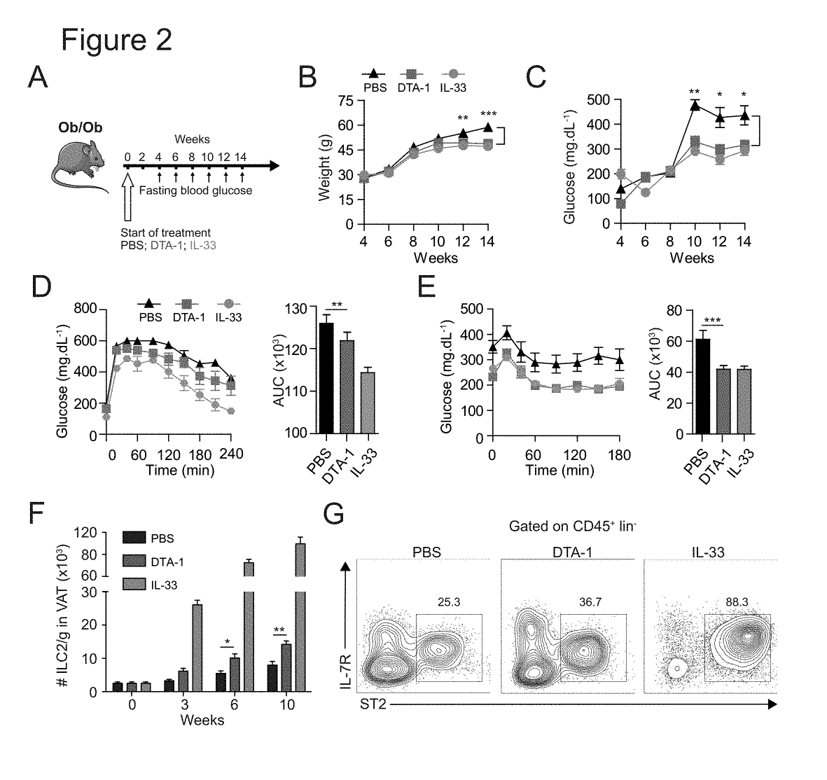

IL-33 induced activation of ILC2s in VAT limits adiposity and insulin resistance in mice fed a high fat diet (HFD). Therefore it was determined if activation of ILC2s through GITR engagement could similarly prevent the development of T2DM in vivo using the mouse model of leptin deficiency Ob/Ob mice fed a regular chow diet, who spontaneously develop severe obesity associated with insulin resistance. A cohort of Ob/Ob mice were treated with intraperitoneal injections of either PBS, DTA-1 (1 mg/mL) or IL-33 (0.5 μg/mL) every four days for 14 weeks (FIG. 2A), and assessed a variety of metabolic parameters. Mice treated with either DTA-1 or IL-33 gained less weight and had lower fasting blood glucose levels as compared to PBS-treated mice (FIGS. 2B and 2C). Additionally, DTA-1 and IL-33 treated mice both showed improvements in glucose tolerance and insulin sensitivity compared to the PBS control mice during intraperitoneal glucose tolerance tests (ip-GTTs) and insulin tolerance tests (ITTs), respectively (FIGS. 2D and 2E). Interestingly, treatment of mice with either DTA-1 or IL-33 resulted in increased numbers of ILC2s in the VAT over time, as compared to control PBS-treated mice. This observed induction of ILC2s in the VAT was higher after IL-33 administration (FIGS. 2F and 2G). Moreover, frequency and numbers of ILC2s in the bone marrow (BM) were also higher in IL-33 and DTA-1 treated mice as compared to control PBS-treated mice (Figure S1). The source of IL-33 and IL-25 responsible for ILC2 activation was also investigated in the context of low grade inflammation metabolic syndrome. Interestingly, we observed that IL-33 and IL-25 were both significantly increased over time by quantitative real time PCR in mice fed a HFD compared to mice fed a chow diet in VAT lysates, suggesting local secretion of IL-33 and IL-25 (Figure S2). Collectively, these observations demonstrate that GITR engagement can limit the onset of spontaneous obesity and improve glucose homeostasis. These results are consistent with previous studies suggesting that IL-33-mediated activation of ILC2s can protect from the development of metabolic syndrome.

### GITR Engagement in ILC2s Prevents Type-2 Diabetes

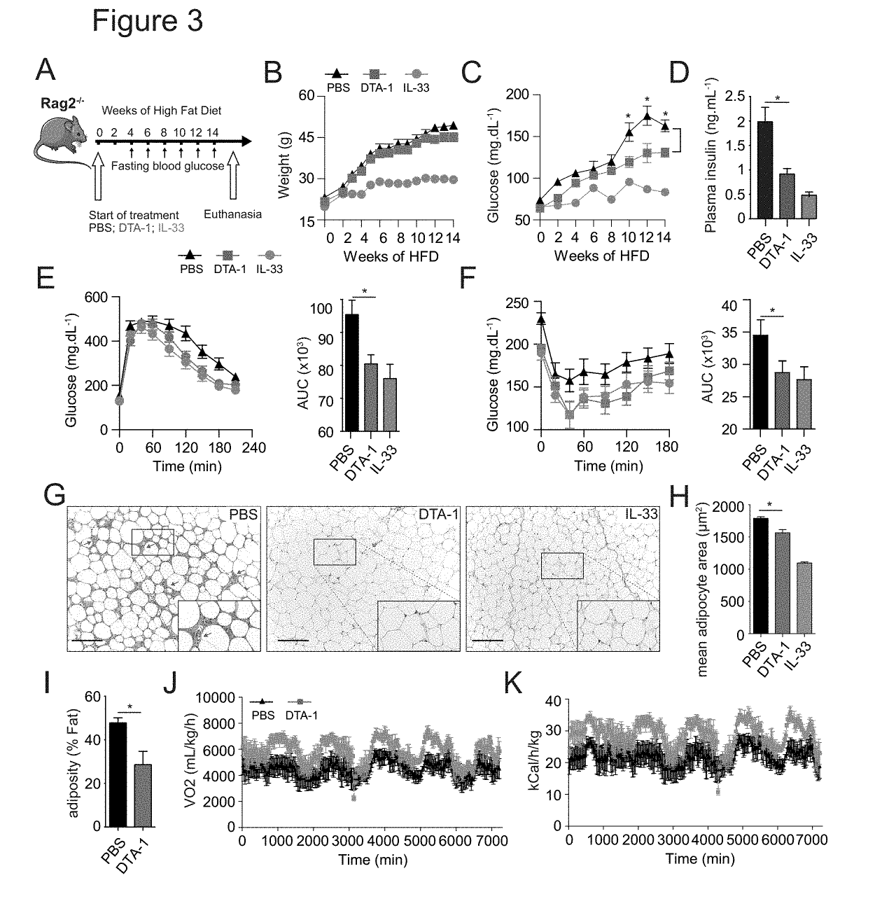

GITR expression is not restricted to ILC2s and is also present on T cells. It was therefore assessed if GITR engagement is effective in Rag2 deficient mice, which lack the adaptive branch of the immune system but still have ILC2s. Therefore, a cohort of Rag2^{-/-} mice were fed a HFD and either treated with PBS, DTA-1 (1 mg/mouse) or IL-33 (0.5 μg/mouse) by intraperitoneal injections every four days for 14 weeks (FIG. 3A). Similar to our results observed in the Ob/Ob mouse model, IL-33 and, to a lesser degree DTA-1 treatment, induced less weight gain compared to PBS-treated mice (FIG. 3B). This protective phenotype was also associated with reduced fasting glucose and insulin concentrations in plasma (FIGS. 3E and 3F), as well as increased glucose tolerance and insulin sensitivity during ip-GTTs and ITTs, respectively (FIGS. 3E and 3F). In addition, we compared the effects of DTA-1 and IL-33 treatment on the VAT structure by histology. In response to DTA-1 and IL-33 treatments, the number of infiltrated leukocytes, the adipocyte size and whole-body adiposity were reduced (FIG. 3G-I) compared with the control group. These data suggest that the protective effects of GITR engagement on glucose homeostasis are independent from the adaptive immune system.

To further understand the mechanisms by which GITR regulates adiposity, DTA-1 and PBS treated Rag2^{-/-} mice were isolated in metabolic cages. Although no difference in food and water intake nor in physical activity was observed (Figure S3), total oxygen consumption (VO₂) and energy utilization (heat) were increased in DTA-1 treated mice (FIGS. 3J and 3K). These results suggest that the metabolic improvements associated with GITR engagement could be mediated, at least in part, through increased oxidative metabolism rather than through effects on caloric intake or caloric expenditure.

### GITR Agonist Induces Th2 Cytokines by ILC2s and Modulates Macrophage Polarization

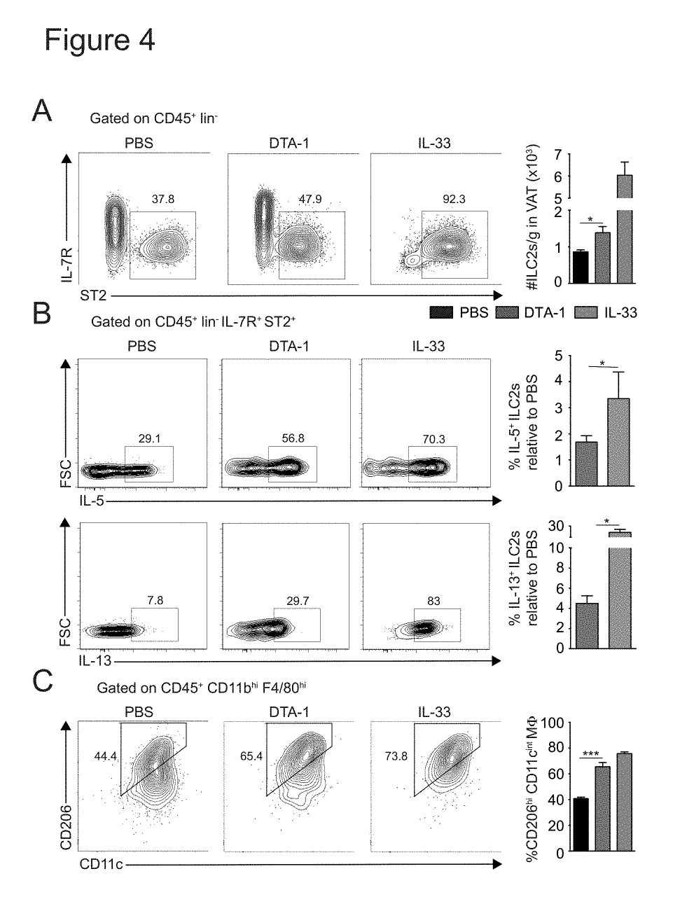

It was previously shown herein that GITR engagement in vitro promotes IL-5 and IL-13 secretion in activated ILC2s (FIG. 1D). These cytokines are often associated with protective effects on the development of T2DM, as IL-13 production promotes an alternatively activated macrophage (AAM) phenotype and IL-5 is required for eosinophil recruitment and activation (von Moltke and Locksley, 2014). Upon activation, eosinophils home to the VAT where they contribute to AAM maintenance and systemic insulin sensitivity. Therefore in vivo intracellular cytokine secretion was measured from VAT-derived ILC2s and M2 macrophage polarization in Rag2^{-/-} mice fed HFD for 14 weeks treated with either DTA-1 or IL-33 (compared to PBS-treated controls). Consistent with previous results in the Ob/Ob mouse model, GITR treatment in Rag2^{-/-} mice increased the number of ILC2s recruited to VAT (FIG. 4A).

Furthermore, the frequencies of ILC2s that secrete IL-5 or IL-13 in the VAT are also increased in IL-33 and DTA-1 treated mice as compared to the control group (FIG. 4B and Figure S4). Interestingly, no effect was observed upon GITR engagement for IL-5 and IL-13 secretion in ILC2s isolated from the VAT of mice fed a chow diet (Figure S4). The frequency of AAMs was increased in both IL-33 and DTA-1 groups in comparison to the PBS treated group. Taken together, these data suggest that GITR engagement drives Th2 cytokine secretion in ILC2s which in turn favors an AAM phenotype in the adipose tissue. These results also demonstrate that this activation of ILC2s from the VAT is dependent on the inflammatory state of the ILC2 environment.

### GITR Agonist Treatment Ameliorates Established Type2 Diabetes

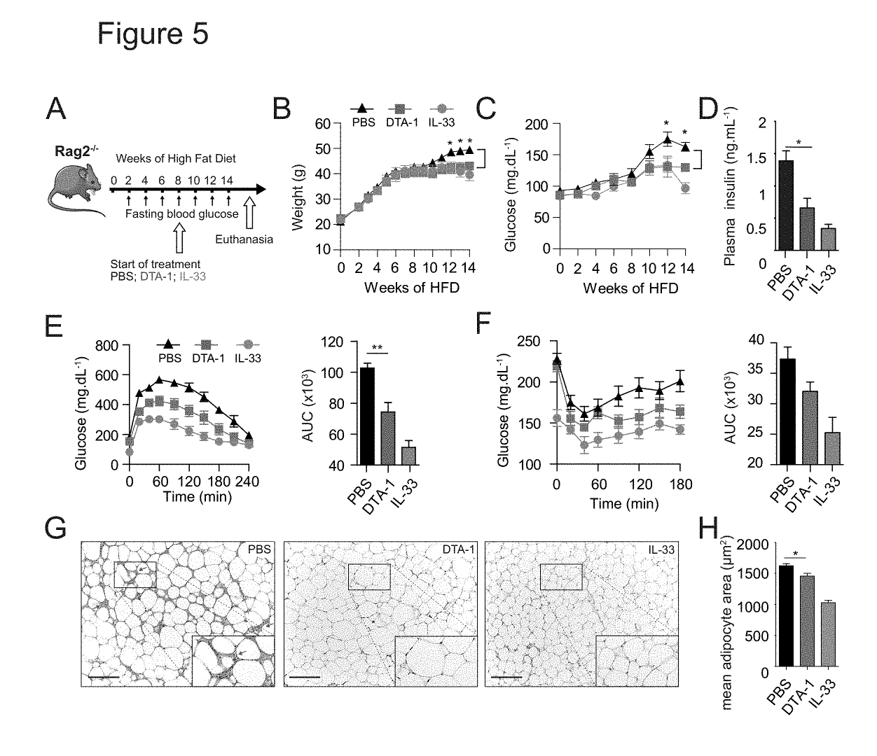

As the findings herein showed that DTA-1 treatment improved the regulation of glucose homeostasis and decreased adiposity in the context of obesity, it was next investigated whether DTA-1 treatment could also have a therapeutic effect on mice with established T2DM. Rag2^{-/-} mice were fed a HFD for 14 weeks; after 8 weeks when T2D is established, mice were then either treated with PBS, DTA-1 (1 mg/mouse) or IL-33 (0.5 μg/mouse) by intraperitoneal injections every four days, as described in FIG. 5A. Similar to our previous results, DTA-1 and IL-33 were associated with less weight gain over time, increased glucose tolerance and insulin sensitivity compared to PBS treated mice (FIGS. 5B-C and 5E-F). Decreased plasma insulin levels were also observed in response to DTA-1 and IL-33 treatment (FIG. 5D). Histological analysis of VAT from DTA-1 and IL-33 treated mice also demonstrated less infiltration of leukocytes and a smaller average adipocyte size compared to PBS-treated mice (FIGS. 5G and 5H). These data indicate that GITR agonist treatment is able to reverse T2D in mice with established metabolic syndrome.

### GITR Engagement on ILC2s is Sufficient to Prevent T2D

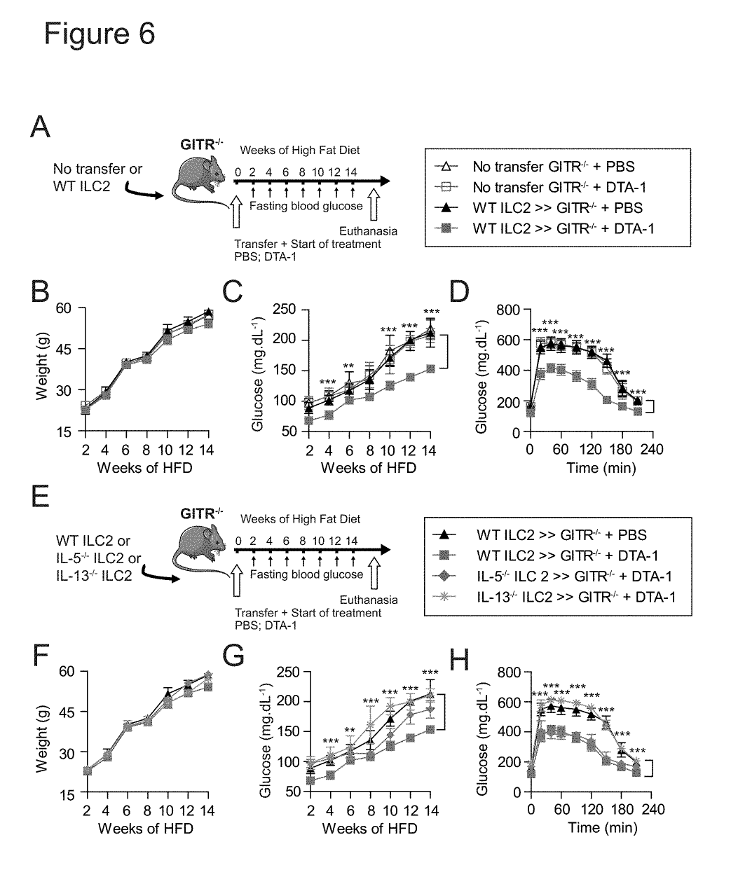

To assess further if engagement of GITR on ILC2s is sufficient to regulate glucose and prevent induction of T2D, GITR^{-/-} mice were adoptively transferred with ILC2s isolated from Wild-Type (WT) mice. After adoptive transfer, mice were treated with PBS or DTA-1 (1 mg/mouse) by intraperitoneal injections every four days, fed an HFD for 14 weeks, and development of T2DM was measured in each group (FIG. 6A). DTA-1 treatment did not have any effect on weight gain or improve the glucose tolerance in GITR^{-/-} mice which did not receive any WT ILC2s (FIG. 6 B-D). However, DTA-1 treatment reduced fasting glycemia and improved glucose tolerance only in GITR^{-/-} mice that were adoptively transferred with WT ILC2s (FIG. 6 B-D). These results demonstrate that the protective effect of DTA-1 treatment is dependent on the expression of GITR on ILC2s. Based on our observations that DTA-1 treatment upregulated intracellular IL-5 and IL-13 expression within VAT ILC2s (FIG. 4), we next investigated whether the protective effect of DTA-1 was mediated by these cytokines. GITR^{-/-} mice were injected with ILC2s isolated either from WT, IL-5^{-/-} or IL-13^{-/-} mice. After adoptive transfer, mice were treated with PBS or DTA-1 by intraperitoneal injections every four days, fed an HFD for 14 weeks, and development of T2DM was measured in each group (FIG. 6E). Although we observed no difference on weight gain between all groups of mice, the protective effect of ILC2s on glucose tolerance at the end of treatment disappeared in mice adoptively transferred with IL-13^{-/-} ILC2s as compared to WT ILC2s (FIGS. 6F and 6H). Furthermore, the protective effect of GITR engagement on fasting glucose during treatment was either partially or completely repressed in mice injected with either IL-5^{-/-} or IL-13^{-/-} ILC2s, respectively (FIG. 6G). Collectively, these results suggest that the protective effects of GITR engagement is dependent on ILC2-derived Th2-cytokine secretion and IL-13 in particular.

### GITR Engagement Induces NF-κB Pathway Signaling in ILC2s

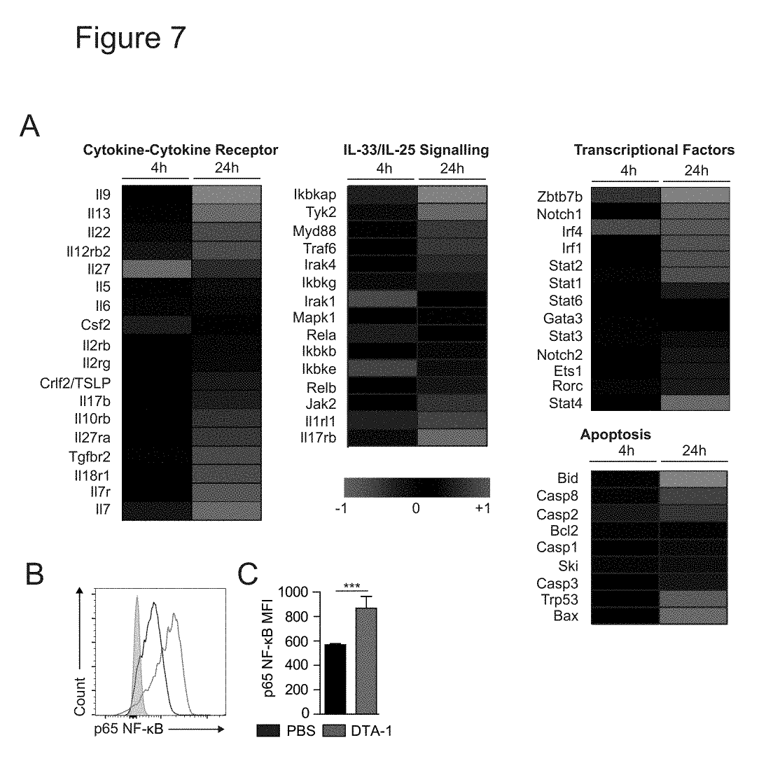

To investigate the molecular mechanisms associated with the protective effects of GITR engagement, we next analyzed the gene expression profile of ILC2s either treated with PBS or DTA-1 (5 μg/mL) for 4 and 24 hours in vitro, by using NanoString technology. NanoString nCounter allows direct measurement of the abundance of transcripts. The evaluated genes were categorized into four panels; genes involved in cytokine signaling pathways, IL-33/IL-25 signaling pathway, transcriptional factors and genes involved in apoptosis (FIG. 7A). As demonstrated in the first panel, expression of IL-9, IL-13, IL-22, IL-27 and IL-5 were all induced after DTA-1 treatment. These results are consistent with our in vitro (FIG. 1D) and in vivo (FIG. 4B) data. Consistent with reports describing NF-κB as being downstream of the activation pathways of GITR (Esparza and Arch, 2004), our gene expression analysis also revealed Rela, which codes for NF-κB p65, as being upregulated after 4 h of DTA-1 treatment (second panel). In the third panel, gene expression of Notch1, Ifr4 and Irf1 is increased in response to GITR engagement whereas Stat4 was inhibited. In the fourth panel, we also observed that Bc12, which is an anti-apoptotic gene, was upregulated in response to GITR engagement. These upregulations were associated with downregulation of the expression of Casp1 and Casp3 genes which play a central role in the execution-phase of cell apoptosis. Furthermore, the expression of the pro-apoptotic genes BAX, Trp53—also known as the tumor suppressor p53- and Ski were also decreased. Upregulation of NF-κB p65 on ILC2s in the presence of DTA-1 was further confirmed at the protein level by flow cytometry (FIGS. 7B and 7C). Collectively, these results demonstrate that GITR engagement highly induces cytokine secretion, anti-apoptotic pathways and thus promotes activated ILC2 survival.

### GITR is Expressed on Human ILC2s and Augments Th2-Cytokine Production

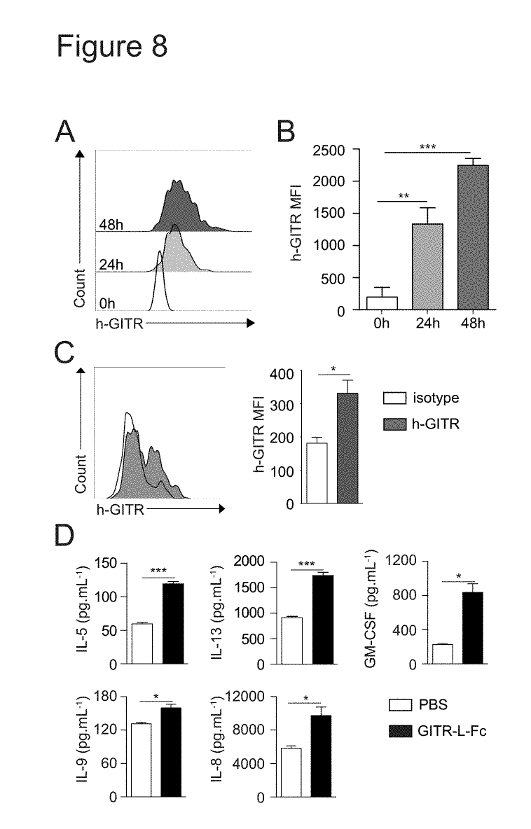

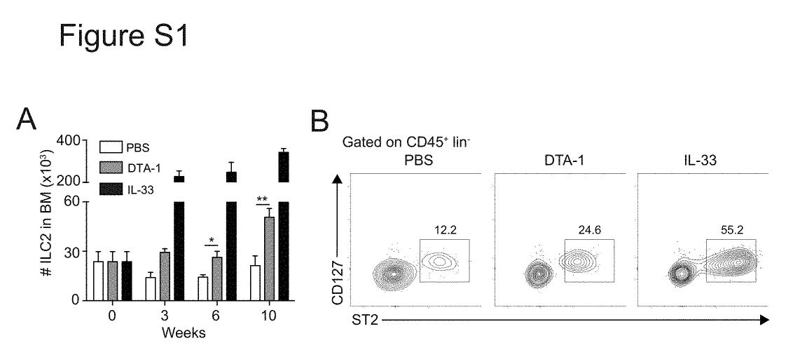

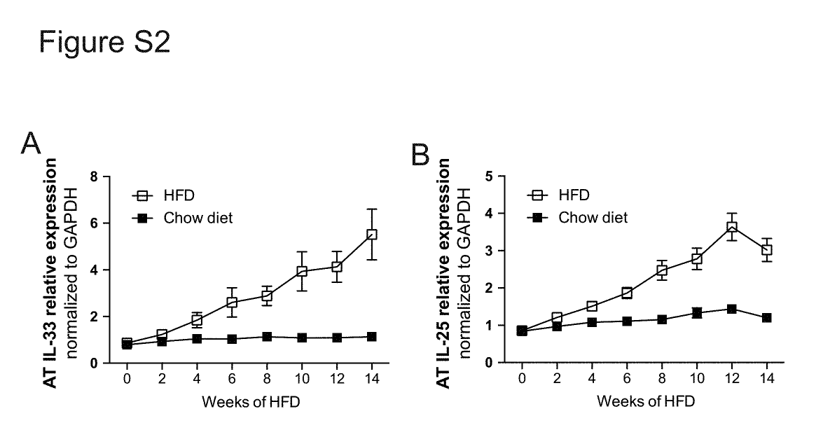

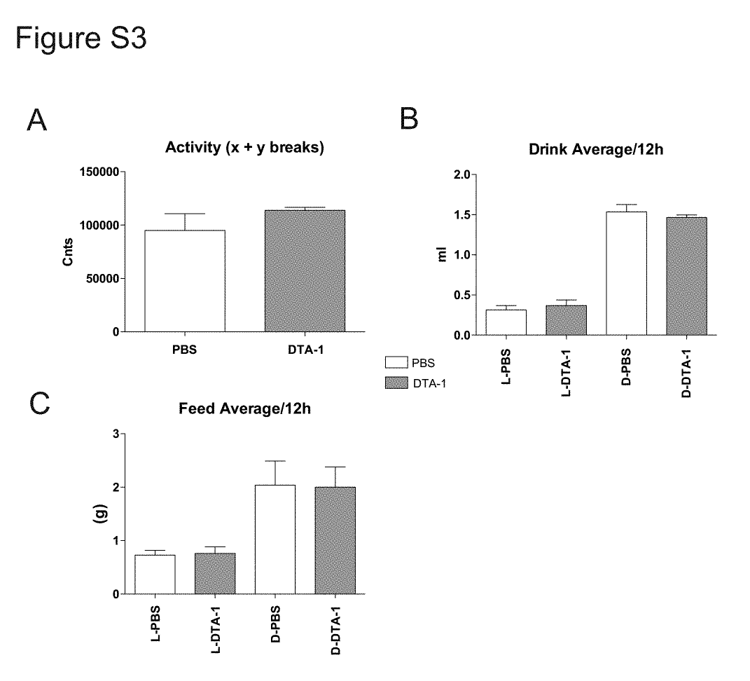

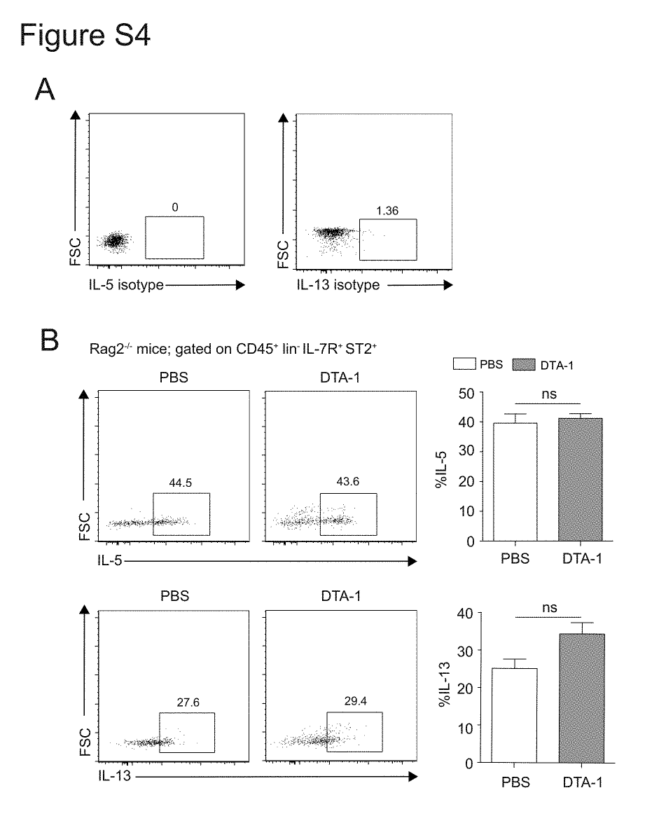

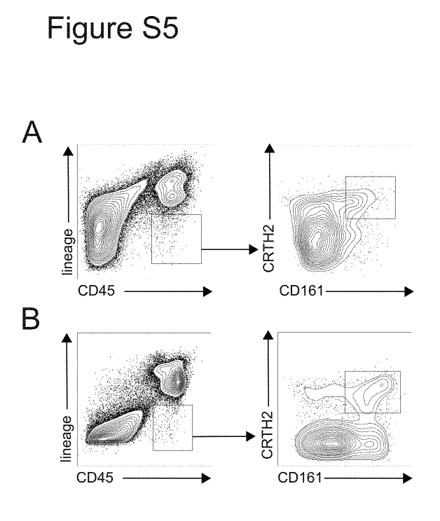

We next explored whether human ILC2s express GITR and whether GITR engagement could play a crucial role in the activation and function of human ILC2s. Purified peripheral blood ILC2s from healthy donors were cultured with recombinant human (rh) IL-2, rhIL-7 and rhIL-33 and plate bound GITR-L-Fc or isotype control for 0, 24, and 48 hours (Figure S5 panel A). We found that GITR was induced on human ILC2s in a time-dependent manner. Indeed, the longer the incubation in presence of GITR-L-Fc, the stronger was GITR expression on the cell surface (FIGS. 8A and 8B). In line with our in vitro observation, human GITR was also expressed on the surface of VAT-derived ILC2s from healthy subjects (FIG. 8C). Considering that upon activation ILC2s secrete higher levels of Th2 cytokines such as IL-5 and IL-13, we also measured by Luminex Th2 cytokines on the corresponding supernatant of human blood ILC2s cultured on the GITR-L-Fc plate bound for 24 hours. In line with our data obtained with murine ILC2s, in response to GITR engagement human ILC2s secrete high amounts of IL-5, IL-13, GM-CSF, IL-8, and IL-9 in presence of rhIL-33 (FIG. 8D). Taken together, these results show that human ILC2s express GITR and that GITR engagement induces Th2 cytokine secretion on rhIL-33 activated ILC2s, suggesting a co-stimulatory role of the GITR receptor in human ILC2s similar to that observed in mice.

### Example 3—Summary and Discussion

It was determined herein that GITR engagement on ILC2s with the specific agonist DTA-1 induces Th2-cytokine secretion in activated ILC2s but has no effect on naïve ILC2s. Importantly, it was determined that GITR engagement is protective against T2DM onset and can also ameliorate established T2DM. By inducing Th2-cytokine secretion in activated ILC2s, GITR engagement modulates macrophage polarization which in turn favors insulin sensitivity. These results suggest that GITR engagement can be therapeutic against T2DM and also that GITR acts as an immune checkpoint for activated ILC2s.

Further, as shown herein, activation of GITR signaling pathway results in enhanced cytokine secretion, increased ILC2 recruitment and induction of anti-apoptotic genes suggesting that GITR engagement also promotes cell survival. These results clearly illustrate that in the context of metabolic syndrome, ILC2s are primed and readily respond to GITR costimulation. This primary signal is critical for GITR signaling as GITR engagement has no effect on naïve ILC2s. The increased concentration of IL-33 and IL-25 in the VAT observed herein, in association with the low grade inflammation linked to metabolic syndrome, constitutes a primary signal vital for ILC2 activation. Furthermore, the absence of secretion activation in VAT ILC2s in response to GITR engagement in lean mice also highlights the requirement for a primary signal in GITR signaling. Accordingly, GITR can act as an immune checkpoint capable of costimulating activated ILC2s.

Also as described herein, in response to GITR engagement of ILC2s the number of alternatively activated macrophages (AAMs) in the adipose tissue was increased. This recruitment of AAMs was associated with decreased insulin resistance, increased glucose tolerance and reduced adiposity. These findings are consistent with metabolic underpinnings of anti-inflammatory mechanisms in other obesity studies. Also as described herein, GITR engagement induces Th2-cytokine secretion in activated ILC2s, resulting in increased numbers of AAMs in the adipose tissue and improvement of glucose tolerance in mice. It was further validated that the protective role of GITR engagement was dependent on Th2-cytokine secretion by using transgenic knockout mouse models in conjunction with adoptive transfer studies. Interestingly, the protective effect of GITR engagement on insulin resistance was synergistic with, or improved markedly by, the presence of IL-13. The secretion of IL-13 has been previously described to polarize macrophages towards an “alternatively activated” phenotype, which in turn helps to maintain glucose homeostasis and dampen inflammation. It is noteworthy to mention that GITR engagement demonstrated not only a preventive role against the onset of T2DM but also therapeutic effects, as the binding of the GITR specific agonist on ILC2s improved insulin sensitivity of diabetic mice.

The results herein also address the molecular mechanism for GITR engagement on ILC2s since it was demonstrated that expression of the activated NF-κB p65 subunit at both the mRNA and protein levels was induced in response to DTA-1 treatment. Collectively, these results suggest that GITR engagement can repress a key pro-inflammatory signaling pathway mediated via NF-κB.

It was further investigated whether this protective effect of GITR engagement was relevant in human cells. It was observed that upon activation ILC2s express GITR in a time-dependent manner resulting in an induction of Th2-cytokine secretion. In accordance with the effects seen in murine ILC2s, specific GITR agonist significantly stimulated ILC2s suggesting a costimulatory role of the GITR receptor in human ILC2s. Taken in their entirety, these results suggest that activating GITR signaling pathway can be used as an effective therapeutic strategy to prevent and improve T2DM.

In conclusion, the data presented herein is the first report to demonstrate GITR is expressed on ILC2s, and upon engagement with a GITR agonist, results in Th2-cytokine secretion, altogether demonstrating a co-stimulatory immune checkpoint role for GITR. Strikingly, it was demonstrated that GITR engagement improves glucose tolerance and insulin sensitivity not only in a preventive manner, but also in a therapeutic manner. These results suggest a protective role of GITR signaling in metabolic syndromes and teach that a GITR agonist can be used as a novel preventive and therapeutic agent for regulating T2DM, and related disorders.

### Example 4—Certain Embodiments

A1. A method of preventing or treating diabetes, or a related disorder, in a subject comprising: providing a subject having, suspected of having, or at risk of having diabetes, or a related disorder, and administering a therapeutically effective amount of a Glucocorticoid-Induced Tumor Necrosis Factor Receptor (GITR) agonist.
A2. A method of regulating blood glucose levels in a subject comprising administering a therapeutically effective amount of a GITR agonist to the subject.
A3. A method of treating or preventing insulin resistance in a subject comprising: providing a subject having, suspected of having, or at risk of having insulin resistance and administering a therapeutically effective amount of a GITR agonist to the subject.
A4. A method of improving glucose tolerance and/or insulin sensitivity in a subject comprising administering a therapeutically effective amount of a GITR agonist to the subject.
A5. The method of any one of embodiments A1 to A4, wherein the GITR agonist specifically binds to GITR.
A6. The method of any one of embodiments A1 to A5, wherein the GITR agonist activates cell signaling through GITR and/or activates NF-kB.
A7. The method of any one of embodiments A1 to A6, wherein the GITR agonist is an antibody, antigen binding fragment thereof, or antibody-like agent that specifically binds to GITR.
A8. The method of embodiment A7, wherein the antibody is a monoclonal antibody.
A9. The method of any one of embodiments A1 to A8, wherein the GITR agonist comprises DTA-1, a humanized version of DTA-1 or a chimeric versions of DTA-1.
A10. The method of any one of embodiments A1 to A6, wherein the GITR agonist comprises a GITR ligand (GITRL), or a variant or derivative thereof.
A11. The method of any one of embodiments A1 to A10, wherein the subject is a mammal. A12. The method of any one of embodiments A1 to A10, wherein the mammal is a human or a mouse.
A13. A method of preventing, or preventing the onset of, or treating diabetes, or a related disorder, in a subject comprising contacting a type-2 innate lymphoid cell with a GITR agonist.
A14. A method of regulating blood glucose levels in a subject comprising contacting a type-2 innate lymphoid cell with a GITR agonist.
A15. A method of treating or preventing insulin resistance in a subject comprising contacting a type-2 innate lymphoid cell with a GITR agonist.
A16. A method of improving glucose tolerance and/or insulin sensitivity in a subject comprising contacting a type-2 innate lymphoid cell with a GITR agonist.
A17. A method of inducing production of, and/or secretion of, one or more Th2-cytokines from a type-2 innate lymphoid cell comprising contacting the type-2 innate lymphoid cell with a GITR agonist.
A18. A method of inducing production of, and/or secretion of, IL-5, IL-13, GM-CSF, IL-6 or IL-9 in or from a type-2 innate lymphoid cell comprising contacting the type-2 innate lymphoid cell with a GITR agonist.
A19. A method of activating a type-2 innate lymphoid cell comprising contacting the type-2 innate lymphoid cell with a GITR agonist.
A20. A method of inducing NF-kB pathway signaling in a type-2 innate lymphoid cell comprising contacting the type-2 innate lymphoid cell with a GITR agonist.
A21. A method of increasing the amount or cell numbers of a type-2 innate lymphoid cell in adipose tissue comprising contacting the a type-2 innate lymphoid cell with a GITR agonist.
A22. A method of protecting a subject against obesity-induced metabolic disturbances comprising contacting a type-2 innate lymphoid cell with a GITR agonist.
A23. A method of modulating macrophage polarization comprising contacting a type-2 innate lymphoid cell with a GITR agonist.
A24. A method of ameliorating established diabetes, or a related disorder in a subject comprising contacting a type-2 innate lymphoid cell with a GITR agonist.
A25. The method of embodiment A20, wherein the NF-kB signaling is anti-inflammatory.
A26. The method of embodiment A19, wherein the activating comprises increasing production and/or secretion of TH2 cytokines.
A27. The method of embodiment A14, wherein the regulating of blood glucose levels in the subject comprising maintaining blood glucose levels within a normal healthy range, lowering blood glucose levels to a normal healthy range or preventing elevation of blood glucose levels to an abnormal unhealthy range.
A28. The method of embodiment A21, wherein the adipose tissue is visceral adipose tissue (VAT).
A29. The method of anyone of embodiments A13 to A28, wherein the GITR agonist specifically binds to a GITR.
A30. The method of anyone of embodiments A13 to A29, wherein the GITR agonist activates cell signaling through the GITR and/or activates NF-kB.
A31. The method of embodiment A29 or A30, wherein the GITR is expressed on, or located on, the surface of the type-2 innate lymphoid cell.
A32. The method of any one of embodiments A13 to A31, wherein the GITR agonist is an antibody, antigen binding fragment thereof, or antibody-like agent that specifically binds to GITR.
A33. The method of embodiment A32, wherein the antibody is a monoclonal antibody.
A34. The method of any one of embodiments A13 to A33, wherein the GITR agonist comprises DTA-1, a humanized version of DTA-1 or a chimeric version of DTA-1.
A35. The method of any one of embodiments A13 to A31, wherein the GITR agonist comprises a GITR ligand (GITRL), or a variant or derivative thereof.
A36. The method of any one of embodiments A13 to A35, wherein the type-2 innate lymphoid cell is located in a subject.
A37. The method of embodiment A36, wherein the subject is a mammal.
A38. The method of embodiment A36 or A37, wherein the mammal is a human or a mouse.
A39. The method of any one of embodiments A36 to A38, wherein the subject has, is suspected of having, or is at risk of having Type-1 diabetes, Type-2 Diabetes, Type-2 Diabetes Mellitus, insulin resistance, elevated blood glucose levels, obesity, or a combination thereof.
A40. The method of any one of embodiments A36 to A30, wherein the method comprises administering a therapeutically effective amount of the GITR agonist to the subject.
A41. The method of any one of embodiments A1 to A35, where the type-2 innate lymphoid cell is obtained from, or derived from a subject.
A42. The method of any one of embodiments A1 to A35, and 41, where the method is conducted in vitro, ex vivo or in vivo.
A43. The method of any one of embodiments A13 to A42, wherein the method further comprises contacting the type-2 innate lymphoid cell with IL-33 or IL-25.
A44. The method of any one of embodiments A13 to A43, wherein the method further comprises contacting the type-2 innate lymphoid cell with IL-2 or IL-7.
A45. The method of any one of embodiments A13 to A44, wherein the type-2 innate lymphoid cell is a mammalian cell.
A46. The method of any one of embodiments A13 to A45, wherein the type-2 innate lymphoid cell is a human cell.
A47. The method of any one of embodiments A1 to A46, wherein the diabetes is Type-2 Diabetes Mellitus.

### Example 5

Sequences

```
huGIRT-Fc DNA sequence - SEQ ID NO: 1
ATGGCACAGCACGGGGCGATGGGCGCGTTTCGGGCCCTGTGCGGCCTGGCGCTGCT
GTGCGCGCTCAGCCTGGGTCAGCGCCCCACCGGGGGTCCCGGGTGCGGCCCTGGGC
GCCTCCTGCTTGGGACGGGAACGGACGCGCGCTGCTGCCGGGTTCACACGACGCGC
TGCTGCCGCGATTACCCGGGCGAGGAGTGCTGTTCCGAGTGGGACTGCATGTGTGTC
CAGCCTGAATTCCACTGCGGAGACCCTTGCTGCACGACCTGCCGGCACCACCCTTGT
CCCCCAGGCCAGGGGGTACAGTCCCAGGGGAAATTCAGTTTTGGCTTCCAGTGTATC
GACTGTGCCTCGGGGACCTTCTCCGGGGGCCACGAAGGCCACTGCAAACCTTGGAC
AGACTGCACCCAGTTCGGGTTTCTCACTGTGTTCCCTGGGAACAAGACCCACAACGC
TGTGTGCGTCCCAGGGTCCCCGCCGGCAGAGGCGGCCGCAGACAAAACTCACACAT
GCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTG
GTGGACGTGAGCCACGAAGACCCTGAGGTCAAGTTCAACTGGTACGTGGACGGCGT
GGAGGTGCATAATGCCAAGACAAAGCCGCGGGAGGAGCAGTACAACAGCACGTAC
CGGGTGGTCAGCGTCCTCACCGTCCTGCACCAGGACTGGCTGAATGGCAAGGAGTA
CAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGGGAT
GAGCTGACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAAGCTCACCGTG
GACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGTAAATGA
    1-75 Signal Sequence
  76-483 human GITR extracellular Domain
493-1176 human Fc region
huGitr-Fc AA sequence - SEQ ID NO: 2
MAQHGAMGAFRALCGLALLCALSLGQRPTGGPGCGPGRLLLGTGTDARCCRVHTTRC
CRDYPGEECCSEWDCMCVQPEFHCGDPCCTTCRHHPCPPGQGVQSQGKFSFGFQCIDC
ASGTFSGGHEGHCKPWTDCTQFGFLTVFPGNKTHNAVCVPGSPPAEAAADKTHTCPPCP
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK*
    1-19 Signal peptide
  20-161 human GITR extracellular Domain
163-392 human Fc region
Fc-huGITRL DNA Sequence - SEQ ID NO: 3
ATGGACTTTGGGCTCAGCTTCATTTTCCTTGCCCTTATTTTAAAAGGTGTCCAGTGTG
ACAAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTG
AATGGCAAGGAGTACAAGTGCAAGGTCTCCAACAAAGCCCTCCCAGCCCCCATCGA
GAAAACCATCTCCAAAGCCAAAGGGCAGCCCCGAGAACCACAGGTGTACACCCTGC
CCCCATCTCGGGATGAGCTGACCAAGAACCAGGTCAGCCTGACCTGCCTGGTCAAA
GGCTTCTATCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCATGCTCCG
TGATGCATGAGGCTCTGCACAACCACTACACGCAGAAGAGCCTCTCCCTGTCCCCGG
GTGCCACTGCTAAGGAGCCCTGTATGGCTAAGTTTGGACCATTACCCTCAAAATGGC
AAATGGCATCTTCTGAACCTCCTTGCGTGAATAAGGTGTCTGACTGGAAGCTGGAGA
TACTTCAGAATGGCTTATATTTAATTTATGGCCAAGTGGCTCCCAATGCAAACTACA
ATGATGTAGCTCCTTTTGAGGTGCGGCTGTATAAAAACAAAGACATGATACAAACTC
TAACAAACAAATCTAAAATCCAAAATGTAGGAGGGACTTATGAATTGCATGTTGGG
GACACCATAGACTTGATATTCAACTCTGAGCATCAGGTTCTAAAAAATAATACATAC
TGGGGTATCATTTTACTAGCAAATCCCCAATTCATCTCCTAG
    1-57 Signal sequence
  58-735 human Fc
739-1116 human GitrL extracellular domain
huGitrL AA sequence - SEQ ID NO: 4
MDFGLSFIFLALILKGVQCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNH
YTQKSLSLSPGATAKEPCMAKFGPLPSKWQMASSEPPCVNKVSDWKLEILQNGLYLIYG
QVAPNANYNDVAPFEVRLYKNKDMIQTLTNKSKIQNVGGTYELHVGDTIDLIFNSEHQV
LKNNTYWGIILLANPQFIS*
    1-19 Signal peptide
  20-245 human Fc
247-372 human GitrL extracellular domain
Fc-muGirtL DNA sequence - SEQ ID NO: 5
ATGGACTTTGGGCTCAGCTTCATTTTCCTTGCCCTTATTTTAAAAGGTGTCCAGTGTG
ACAAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAGCAG
TACAACAGCACGTACCGGGTGGTCAGCGTCCTCACCGTCCTGCACCAGGACTGGCTG
AATGGCAAGGAGTACAAGTGCAAGGTCTCCAACAAAGCCCTCCCAGCCCCCATCGA
GAAAACCATCTCCAAAGCCAAAGGGCAGCCCCGAGAACCACAGGTGTACACCCTGC
CCCCATCCCGGGATGAGCTGACCAAGAACCAGGTCAGCCTGACCTGCCTGGTCAAA
GGCTTCTATCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCATGCTCCG
TGATGCATGAGGCTCTGCACAACCACTACACGCAGAAGAGCCTCTCCCTGTCCCCGG
GTGCCTCACTCAAGCCAACTGCCATCGAGTCCTGCATGGTTAAGTTTGAACTATCAT
CCTCAAAATGGCACATGACATCTCCCAAACCTCACTGTGTGAATACGACATCTGATG
GGAAGCTGAAGATACTGCAGAGTGGCACATATTTAATCTACGGCCAAGTGATTCCTG
TGGATAAGAAATACATAAAAGACAATGCCCCCTTCGTAGTACAGATATATAAAAAG
AATGATGTCCTACAAACTCTAATGAATGATTTTCAAATCTTGCCTATAGGAGGGGTT
TATGAACTGCATGCTGGAGATAACATATATCTGAAGTTCAACTCTAAAGACCATATT
CAGAAAACTAACACATACTGGGGGATCATCTTAATGCCTGATCTACCATTCATCTCT
TAG
    1-57 Signal sequence
  58-735 human Fc
739-1116 murine GitrL extracellular domain
Fc-muGitrL AA sequence - SEQ ID NO: 6
MDFGLSFIFLALILKGVQCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNH
YTQKSLSLSPGASLKPTAIESCMVKFELSSSKWHMTSPKPHCVNTTSDGKLKILQSGTYL
IYGQVIPVDKKYIKDNAPFVVQIYKKNDVLQTLMNDFQILPIGGVYELHAGDNIYLKFNS
KDHIQKTNTYWGIILMPDLPFIS*
    1-19 Signal peptide
  20-245 human Fc
247-372 murine GitrL extracellular domain
```

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation, and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

1. A method of preventing or treating diabetes, or a related disorder, in a subject comprising: providing a subject having, suspected of having, or at risk of having diabetes, or a related disorder; and administering a therapeutically effective amount of a Glucocorticoid-Induced Tumor Necrosis Factor Receptor (GITR) agonist to the subject.

2. The method of claim 1, wherein the diabetes is selected from Type-1 diabetes, Type-2 Diabetes, and Type-2 Diabetes Mellitus, or the related disorder is selected from insulin resistance, elevated blood glucose levels, and obesity.

3. The method of claim 1, wherein the method comprises regulating blood glucose levels in the subject or improving glucose tolerance and/or insulin sensitivity in the subject.

4. The method of claim 1, wherein the GITR agonist is an antibody, antigen binding fragment thereof, or antibody-like agent that specifically binds to GITR, and activates cell signaling or NF-kB through the GITR.

5. The method of claim 4, wherein the GITR agonist is a monoclonal antibody.

6. The method of claim 4, wherein antibody comprises DTA-1, or a chimeric or humanized version of DTA-1.

7. The method of claim 1, wherein the method comprises contacting a type-2 innate lymphoid cell with the GITR agonist.

8. The method of claim 1, wherein the method comprises treating or preventing insulin resistance in a subject, or improving glucose tolerance and/or insulin sensitivity in a subject comprising contacting a type-2 innate lymphoid cell with the GITR agonist.

9. The method of claim 7, further comprising inducing production of, and/or secretion of, one or more Th2-cytokines, or a cytokine selected from IL-5, IL-13, GM-CSF, IL-6 or IL-9, from the type-2 innate lymphoid cell.

10. The method of claim 7, further comprising activating the type-2 innate lymphoid cell or inducing NF-kB pathway signaling in the type-2 innate lymphoid cell.

11. The method of claim 7, further comprising increasing an amount or cell number of the type-2 innate lymphoid cell.

12. A method of protecting a subject against obesity-induced metabolic disturbances comprising administering a GITR agonist to the subject.

13. The method of claim 12, further comprising contacting a type-2 innate lymphoid cell of the subject with a GITR agonist.

14. The method of claim 12, further comprising modulating macrophage polarization.

15. The method of claim 12, wherein the GITR agonist is an antibody, antigen binding fragment thereof, or antibody-like agent that specifically binds to GITR, and activates cell signaling or NF-kB through the GITR.

16. The method of claim 12, wherein the GITR agonist is a monoclonal antibody or antigen binding fragment thereof, that specifically binds to human GITR.

17. The method of claim 15, wherein the antibody comprises DTA-1, or a chimeric or humanized version of DTA-1.
